(19) United States
(12) Patent Application Publication
(10) Pub. No.: US 2011/0281896 A1
(43) Pub. Date: Nov. 17, 2011

(54) OPTICALLY PURE QUINAZOLINE COMPOUNDS

(75) Inventors: Jiadeng Tang, Shanghai (CN); Xuesong Wu, Shanghai (CN)

(73) Assignee: Jiangsu Hansoh Pharmaceutical Co. Ltd

(21) Appl. No.: 13/145,956

(22) PCT Filed: Jan. 7, 2010
(86) PCT No.: PCT/CN10/00027

§ 371 Date: Jul. 22, 2011

(30) Foreign Application Priority Data
Jan. 23, 2009 (CN) 200910008526.3

(51) Int. Cl. A61K 31/517; A61P 17/06; C07D 405/14; A61P 35/00; C07D 405/10; C07D 417/10

(52) U.S. Cl. 514/266.23; 544/293; 544/284; 514/266.24; 514/266.2

(57) ABSTRACT

Optical pure quinazoline compounds, especially compounds of the general formula (I), wherein R¹ and Y are defined as the specification, preparation methods of them, pharmaceutical compositions containing them and their uses are provided. Compounds of the general formula (VII), which are intermediates in the synthesis of the compounds of the general formula (I), wherein Ar, R², R³, m, n, T and carbon atom with * are defined as the specification, are also provided.

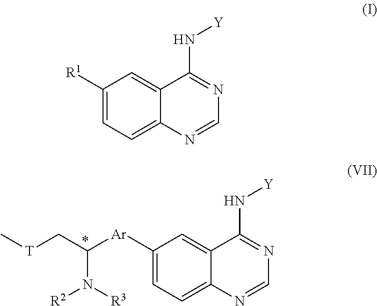

### FIELD OF THE INVENTION

The present invention relates to a series of novel optically pure quinazoline compounds, the processes for preparation, the pharmaceutical composition and the use thereof. The present invention also relates to the intermediates in the synthesis of the novel optically pure quinazoline compounds.

### BACKGROUND OF THE INVENTION

Protein-tyrosine kinases catalyze phosphorylation of specific tyrosyl residues in various proteins that are relevant to the regulation of cell growth and differentiation. Protein-tyrosine kinase can be broadly divided into receptor kinases (such as EGFr, c-erbB-2, c-met, tie-2, PDGFr, FGFr) and non-receptor kinases (such as c-src, lck zap70). It has shown that many protein-tyrosine kinases could be activated unproperly or uncontrollably, and the anomalous activation caused by over-expression or mutation will cause uncontrollable cell production.

Abnormal activity of protein-tyrosine kinase such as c-erbB-2, c-src, c-met, EGFr, PDGFr is related to human malignant tumors. For example, the elevated activity of EGFr is relevant to non-small cell lung cancer, bladder cancer, and head and neck cancer, and the elevated activity of c-erbB-2 is relevant to breast, ovarian, stomach and pancreatic cancers. Therefore, inhibiting protein-tyrosine kinases can provide treatments for the foregoing cancers.

Anomalous protein-tyrosine kinase activity is also related to other diseases, such as: psoriasis, fiber degeneration, atherosclerosis, restenosis, autoimmune diseases, allergies, asthma, etc. It has shown that these diseases can be controlled by acting on tyrosine kinase receptor.

Chinese patent 99803887.3 discloses series of compounds that have inhibition of protein-tyrosine kinase activity. Chinese patent 20081000815 also discloses series of new (racemic) quinazoline compounds, but without the data of their optically pure enantiomers.

### DESCRIPTION OF THE INVENTION

The present invention aims to provide a series of optically pure quinazoline compounds shown in general formula (I), the process of preparation and the use thereof.

The invention also aims to provide a pharmaceutical composition comprising an effective dose of the above-mentioned optically pure quinazoline compounds shown in general formula (I), and their use for treatment of cancers, malignant tumors and psoriasis etc.

The invention also aims to provide intermediates, which are shown in general formula (VII), in the synthesis of the compound of the general formula (I).

The invention discloses compounds of general formula (I):

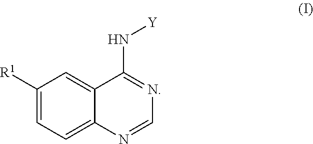

Wherein R¹ represents

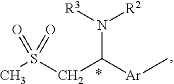

in which Ar is selected from substituted and unsubstituted furan or thiazole. The substituents are selected from halogen atoms, C₁₋₄ alkyl and C₁₋₄ alkoxy, and the number of the substituents is 1 or 2; R² and R³ are independently selected from (1) hydrogen, (2) alkyl, (3) alkenyl, (4) alkynyl, (5) alkoxy, (6) alkoxy alkyl, (7) cycloalkyl, and (8) cycloalkyl alkyl.

Y is selected from phenyl and 1H-indazolyl, substituted independently by R⁴, R⁵ at any position; wherein R⁴ is selected from benzyl, halogenated-, dihalogenated- or trihalogenated benzyl, benzyloxy, halogenated-, and dihalogenated- or trihalogenated benzyloxy.

R⁵ is selected from hydrogen, hydroxy, halogen atoms, C₁₋₄ alkyl, C₁₋₄ alkoxy, amino, cyano, and trifluoromethyl.

The carbon atom with * is a chiral carbon atom, and the compound of formula (I) exists in the form of a single enantiomer or is enriched in one enantiomer of (R) or (S).

In a preferred embodiment of this invention, Ar is selected from unsubstituted furan and unsubstituted thiazole, preferably unsubstituted furan.

In a preferred embodiment of this invention, R² and R³ are independently selected from: (1) hydrogen, (2) C₁₋₄ alkyl, (3) C₂₋₅ alkenyl, (4) C₁₋₄ alkoxy, (5) C₁₋₄ alkoxy C₁₋₄ alkyl, (6) C₃₋₈ cycloalkyl, and (7) C₃₋₈ cycloalkyl-C₁₋₄ alkyl.

In a preferred embodiment of this invention, R⁴ is selected from benzyl, halogenated-benzyl, and halogenated-benzyloxy, preferably halogenated-benzyl or halo-benzyloxy; R⁵ is selected from hydrogen, halogen atoms, C₁₋₄ alkyl, and C₁₋₄ alkoxy.

In a preferred embodiment of this invention, the compound of formula (I) exists in the form of a single enantiomer or is enriched in an enantiomer of (R) or (S). When it exists in the form enriched in (R) or (S), preferably the content of enantiomer of (R) or (S) is ≧90%.

In one embodiment of this invention, the preferred compounds comprise:
(R)—N-(4-(3-fluorobenzyl-oxy)-3-chlorophenyl)-6-(5-(1-(amino)-2-(methylsulfonyl)ethyl)furan-2-yl)quinazolin-4-amine; (compound 105)
(S)—N-(4-(3-fluorobenzyl-oxy)-3-chlorophenyl)-6-(5-(1-(amino)-2-(methylsulfonyl)ethyl)furan-2-yl)quinazolin-4-amine; (compound 92)
(R)—N-(4-(3-fluorobenzyl-oxy)-3-chlorophenyl)-6-(5-(1-(methylamino)-2-(methylsulfonyl)ethyl)furan-2-yl)quinazolin-4-amine; (compound 106)
(R)—N-(4-(3-fluorobenzyl-oxy)-3-chlorophenyl)-6-(5-(1-(ethylamino)-2-(methylsulfonyl)ethyl)furan-2-yl)quinazolin-4-amine; (compound 107)
(R)—N-(4-(3-fluorobenzyl-oxy)-3-chlorophenyl)-6-(5-(1-(propyl amino)-2-(methylsulfonyl)ethyl)furan-2-yl)quinazolin-4-amine; (compound 108)
(R)—N-(4-(3-fluorobenzyl-oxy)-3-chlorophenyl)-6-(5-(1-(cyclopropyl methyl-amino)-2-(methylsulfonyl)ethyl)furan-2-yl)quinazolin-4-amine; (compound 109)
(R)—N-(4-(3-fluorobenzyl-oxy)-3-chlorophenyl)-6-(5-(1-(N,N-dimethyl-amino)-2-(methylsulfonyl)ethyl)-2-yl)quinazolin-4-amine; (compound 110)
(R)—N-(4-(3-fluorobenzyl-oxy)-3-chlorophenyl)-6-(5-(1-(N,N-diethyl-amino)-2-(methylsulfonyl)ethyl)furan-2-yl)quinazolin-4-amine; (compound 111)
(R)—N-(4-(3-fluorobenzyl-oxy)-3-chlorophenyl)-6-(5-(1-(N,N-dipropyl-amino)-2-(methylsulfonyl)ethyl)furan-2-yl)quinazolin-4-amine; (compound 112)
(R)—N-(4-(3-fluorobenzyloxy)-3-chlorophenyl)-6-(5-(1-(N-methyl, N-ethyl-amino)-2-(methylsulfonyl)ethyl)furan-2-yl)quinazolin-4-amine; (compound 113)
(R)—N-(4-(3-fluorobenzyl-oxy)-3-chlorophenyl)-6-(5-(1-(allyl-amino)-2-(methylsulfonyl)ethyl)furan-2-yl)quinazolin-4-amine; (compound 114)
(R)—N-(4-(3-fluorobenzyl-oxy)-3-chlorophenyl)-6-(5-(1-(propargyl-amino)-2-(methylsulfonyl)ethyl)furan-2-yl)quinazolin-4-amine; (compound 115)
(S)—N-(4-(3-fluorobenzyl-oxy)-3-chlorphenyl)-6-(5-(1-(methylamino)-2-(methylsulfonyl)ethyl)furan-2-yl)quinazolin-4-amine; (compound 93)
(S)—N-(4-(3-fluorobenzyl-oxy)-3-chlorophenyl)-6-(5-(1-(ethylamino)-2-(methylsulfonyl)ethyl)furan-2-yl)quinazolin-4-amine; (compound 94)
(S)—N-(4-(3-fluorobenzyl-oxy)-3-chlorophenyl)-6-(5-(1-(propyl-amino)-2-(methylsulfonyl)ethyl)furan-2-yl)quinazolin-4-amine; (compound 95)
(S)—N-(4-(3-fluorobenzyl-oxy)-3-chlorophenyl)-6-(5-(1-(cyclopropyl-methyl-amino)-2-(methylsulfonyl)ethyl)furan-2-yl)quinazolin-4-amine; (compound 96)
(S)—N-(4-(3-fluorobenzyloxy)-3-chlorophenyl)-6-(5-(1-(N,N-dimethyl-amino)-2-(methylsulfonyl)ethyl)furan-2-yl)quinazolin-4-amine; (compound 97)
(S)—N-(4-(3-fluorobenzyl-oxy)-3-chlorophenyl)-6-(5-(1-(N,N-diethyl-amino)-2-(methyl-sulfonyl)ethyl)furan-2-yl)quinazolin-4-amine; (compound 98)
(S)—N-(4-(3-fluorobenzyl-oxy)-3-chlorophenyl)-6-(5-(1-(N,N-dipropyl-amino)-2-(methylsulfonyl)ethyl)furan-2-yl)quinazolin-4-amine; (compound 99)
(S)—N-(4-(3-fluorobenzyl-oxy)-3-chlorophenyl)-6-(5-(1-(N-methyl, N-ethyl-amino)-2-(methylsulfonyl)ethyl)furan-2-yl)quinazolin-4-amine; (compound 100)
(S)—N-(4-(3-fluorobenzyl-oxy)-3-chlorophenyl)-6-(5-(1-(allyl-amino)-2-(methylsulfonyl)ethyl)furan-2-yl)quinazolin-4-amine; (compound 101)
(S)—N-(4-(3-fluorobenzyl-oxy)-3-chlorophenyl)-6-(5-(1-(propargyl-amino)-2-(methylsulfonyl)ethyl)furan-2-yl)quinazolin-4-amine; (compound 102)
(S)—N-(1-(3-fluorobenzyl)-1H-indazol-5-yl)-6-(5-(1-(amino)-2-(methylsulfonyl)ethyl)furan-2-yl)quinazolin-4-amine; (compound 144)
(S)—N-(1-(3-fluorobenzyl)-1H-indazol-5-yl)-6-(5-(1-(methylamino)-2-(methylsulfonyl)ethyl)furan-2-yl)quinazolin-4-amine; (compound 145)
(S)—N-(1-(3-fluorobenzyl)-1H-indazol-5-yl)-6-(5-(1-(ethylamino)-2-(methylsulfonyl)ethyl)furan-2-yl)quinazolin-4-amine; (compound 146)
(S)—N-(1-(3-fluorobenzyl)-1H-indazol-5-yl)-6-(5-(1-(propylamino)-2-(methylsulfonyl)ethyl)furan-2-yl)quinazolin-4-amine; (compound 147)
(S)—N-(1-(3-fluorobenzyl)-1H-indazol-5-yl)-6-(5-(1-(N,N-dimethylamino)-2-(methylsulfonyl)ethyl)furan-2-yl)quinazolin-4-amine; (compound 149)
(S)—N-(1-(3-fluorobenzyl)-1H-indazol-5-yl)-6-(5-(1-(cyclopropyl-methyl-amino)-2-(methylsulfonyl)ethyl)furan-2-yl)quinazolin-4-amine; (compound 148)
(S)—N-(1-(3-fluorobenzyl)-1H-indazol-5-yl)-6-(5-(1-(allyl-amino)-2-(methylsulfonyl)ethyl)furan-2-yl)quinazolin-4-amine; (compound 153) and
(S)—N-(1-(3-fluorobenzyl)-1H-indazol-5-yl)-6-(5-(1-(propargyl-amino)-2-(methylsulfonyl)ethyl)furan-2-yl)quinazolin-4-amine (compound 154).

In the present invention, “enriched in one enantiomer” refers to the content of enantiomer of (R) or (S) is ≧0.60%.

“Alkyl” refers to branched or straight-chain saturated aliphatic hydrocarbon groups. Preferably, branched or straight-chain saturated aliphatic alkyl has a number of carbon atom of 1 to 4, such as methyl, ethyl, propyl, isopropyl, butyl, tert-butyl, isobutyl, etc.

“alkenyl” refers to branched, straight-chain or non-aromatic hydrocarbon ring group which contains at least one carbon-carbon double bonds (—C═C—), such as vinyl, propenyl, allyl, butenyl, cyclohexene, etc.

“Alkynyl” refers to branched, straight-chain or cyclic hydrocarbon group, which contains at least one carbon-carbon triple bond (—C═C—), such as acetenyl, propinyl, butynyl, 3-methyl-butynyl, homopropargyl, propargyl, etc.

“Cycloalkyl” refers to saturated aliphatic hydrocarbon group which contains monocyclic ring. Preferably, cycloalkyl contains 3-8 carbon atoms, such as cyclopropyl, methyl-cyclopropyl, 2,2-dimethyl-cyclobutyl, ethyl-cyclopentyl, cyclohexyl, etc.

“Alkoxy” refers to a group in which straight or branched chain alkyl is connected to oxygen atoms, such as methoxy, ethoxy, propoxy, isopropoxy, butoxy, isobuoxy, tert-butoxy, etc.

“Halogen atoms” refer to fluorine, chlorine, bromine, and iodine atoms.

The invention also aims to provide the preparation process of compounds of general formula (I), comprising the following steps:

1) reacting the compound of general formula (II) with tert-butylsulfinamide to prepare the compound of general formula (III);

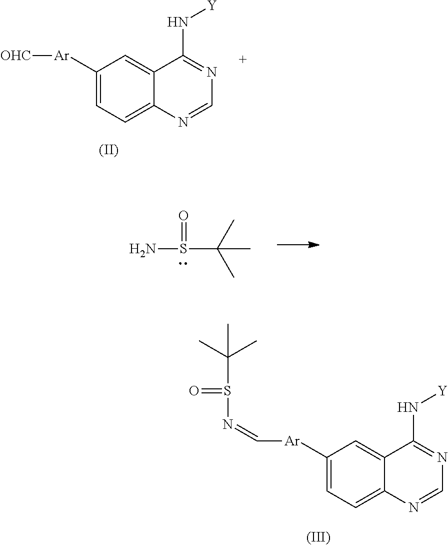

2) reacting the compound of general formula (III) with the compound of general formula (IV) to prepare the compound of general formula (V);

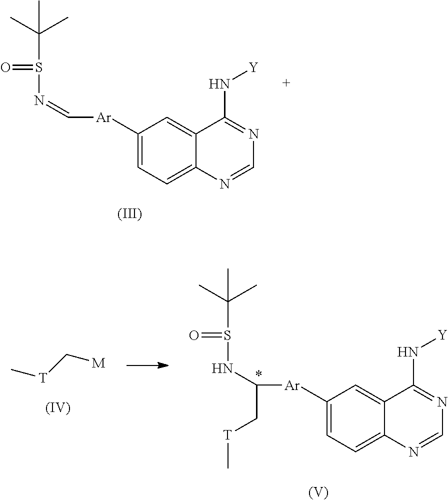

3) generating the compound of general formula (VI) from the compound of general formula (V) in acid condition;

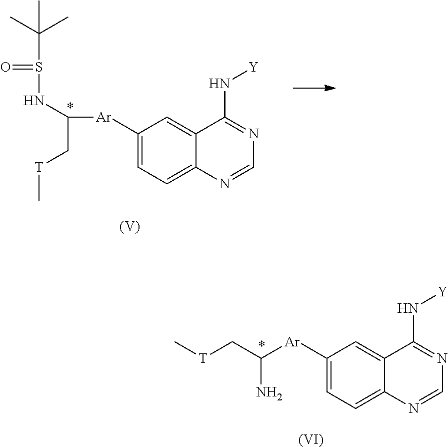

4) reacting the compound of general formula (VI) with reagent R²-L and R³-L to prepare the compound of general formula (VII);

and

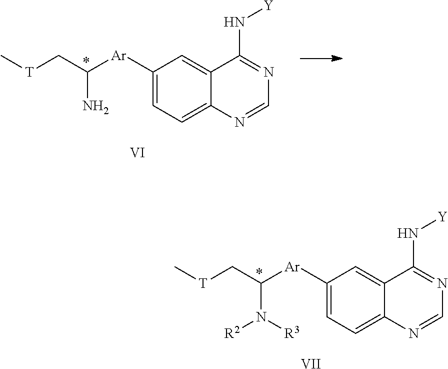

5) reacting the compound of general formula (VII) with oxidant to prepare the compound of general formula (I).

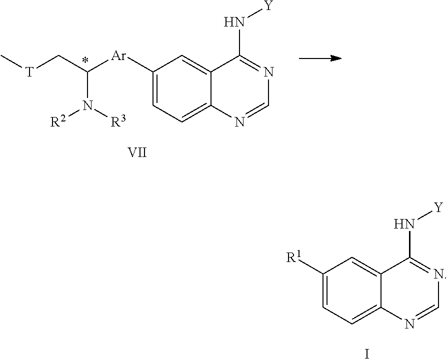

Wherein, R¹, Y, Ar, R², R³, ca^{r}bon atom with * are as defined in formula (I).

T is sulfur atom or sulfinyl.

Tert-butylsulfinamide is optically pure, and exists in the form of a single enantiomer or is enriched in an enantiomer of (R) or (S).

M is alkali metal ion or halogenated-alkaline earth metal ions, selected from Li⁺, Na⁺, K⁺, [MgCl]⁺ or [MgBr]⁺.

L is a leaving group, selected from halogen atom and sulfonyloxyl group.

In the preparation process of general formula (III), the reaction is carried out in the presence of metallic reagents. The metallic reagents comprise tetraethoxy titanium, tetra isopropyl titanate etc., and preferably tetra isopropyl titanate. The reaction temperature is 0-100° C., and preferably 0-50° C.

In the preparation process of general formula (VI), the reaction is carried out under acidic conditions. The acid is selected from hydrochloric acid, sulfuric acid, nitric acid, phosphoric acid, trifluoroacetic acid, and the mixture of the foregoing acids, and preferably hydrochloric acid;

In the preparation process of general formula (I), oxidation of sulfur atom or sulfinyl to sulfonyl is well known by one skilled in the art. The oxidant is selected from chloroperoxybenzoic acid, peracetic acid, hydrogen peroxide and potassium monopersulfate, preferably potassium monopersulfate.

The invention also aims to provide the preparation process of compounds in general formula (I), comprising the following steps:

1) reacting the compound of general formula (II) with tert-butylsulfinamide to prepare the compound of general formula (III);

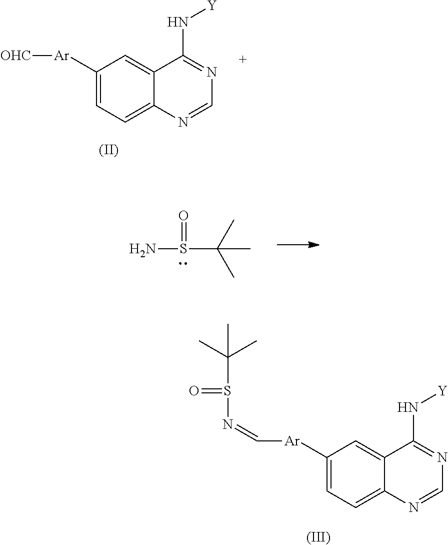

2) reacting the compound of general formula (III) with the compound of general formula (A) to prepare the compound of general formula (B);

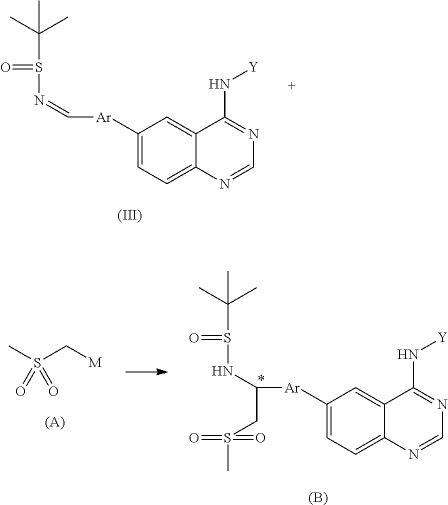

3) generating the compound of general formula (C) from the compound of general formula (B) in acid condition; and

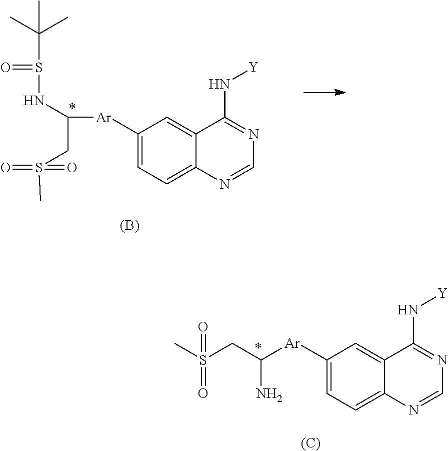

4) reacting the compound of general formula (C) with reagent R²-L or R³-L to prepare the compound of general formula (I).

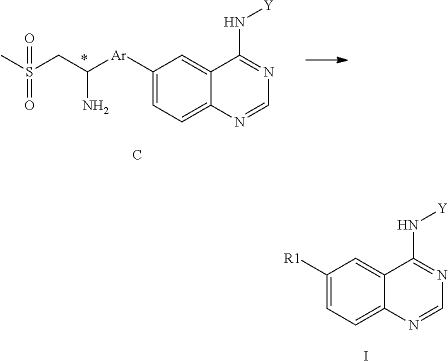

Wherein, R¹, Y, Ar, R², R³, carbon atom with * are as defined in general formula (I). Tert-butylsulfinamide, M, and L are as defined above. In the preparation process of general formula (I), L represents leaving group well-known by person skilled in the art, such as halogen atoms (such as fluorine, chlorine, bromine, iodine atoms), preferably bromine, iodine atoms; Sulfonyloxyl group (such as methylsulfonyloxyl, toluenesulfonyloxyl) etc.

In the preparation process of general formula (C), the reaction is carried out under acidic conditions. The acid is selected from hydrochloric acid, sulfuric acid, nitric acid, phosphoric acid, trifluoroacetic acid and the combinations thereof, and preferably hydrochloric acid.

The reaction of step 4 is carried out under alkaline condition, and the alkaline is selected from inorganic bases (such as sodium bicarbonate, sodium carbonate, potassium carbonate, sodium hydroxide, potassium hydroxide, etc.) and organic bases (such as ethylamine, triethylamine, diisopropylethylamine, etc.).

The invention also aims to provide the intermediate compounds represented by general formula (VII), which are key intermediates for the synthesis of the compounds of general formula (I).

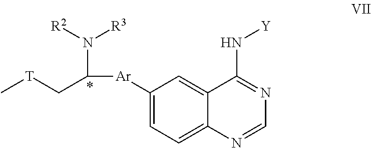

Wherein Y, Ar, R², R³, and the carbon atom with * are as defined in claim 1. T is sulfur atom or sulfinyl.

In the present invention, preferred compounds of general formula (VII) comprise:
(R)—N-(4-(3-fluorobenzyl-oxy)-3-chlorophenyl)-6-(5-(1-(amino)-2-(methylthio)ethyl)furan-2-yl)quinazolin-4-amine; (compound 40)
(R)—N-(4-(3-fluorobenzyl-oxy)-3-chlorophenyl)-6-(5-(1-(methylamino)-2-(methylthio)ethyl)furan-2-yl)quinazolin-4-amine; (compound 41)
(R)—N-(4-(3-fluorobenzyl-oxy)-3-chlorophenyl)-6-(5-(1-(ethylamino)-2-(methylthio)ethyl)furan-2-yl)quinazolin-4-amine; (compound 42)
(R)—N-(4-(3-fluorobenzyl-oxy)-3-chlorophenyl)-6-(5-(1-(propylamino)-2-(methylthio)ethyl)furan-2-yl)quinazolin-4-amine; (compound 43)
(R)—N-(4-(3-fluorobenzyl-oxy)-3-chlorophenyl)-6-(5-(1-(cyclopropyl-methyl-amino)-2-(methylthio)ethyl)furan-2-yl)quinazolin-4-amine; (compound 44)
(R)—N-(4-(3-fluorobenzyl-oxy)-3-chlorophenyl)-6-(5-(1-(N,N-dimethylamino)-2-(methylthio)ethyl)furan-2-yl)quinazolin-4-amine; (compound 45)
(R)—N-(4-(3-fluorobenzyl-oxy)-3-chlorophenyl)-6-(5-(1-(N,N-diethylamino)-2-(methylthio)ethyl)furan-2-yl)quinazolin-4-amine; (compound 46)
(R)—N-(4-(3-fluorobenzyl-oxy)-3-chlorophenyl)-6-(5-(1-(N,N-dipropyl-amino)-2-(methylthiolethyl)furan-2-yl)quinazolin-4-amine; (compound 47)
(R)—N-(4-(3-fluorobenzyl-oxy)-3-chlorophenyl)-6-(5-(1-(N-methyl, N-ethyl-amino)-2-(methylthio)ethyl)furan-2-yl)quinazolin-4-amine; (compound 48)
(R)—N-(4-(3-fluorobenzyl-oxy)-3-chlorophenyl)-6-(5-(1-(allyl-amino)-2-(methylthio)ethyl)furan-2-yl)quinazolin-4-amine; (compound 49)
(R)—N-(4-(3-fluorobenzyl-oxy)-3-chlorophenyl)-6-(5-(1-(propargylamino)-2-(methylthio)ethyl)furan-2-yl)quinazolin-4-amine; (compound 50)
(R)—N-(4-(3-fluorobenzyl-oxy)-3-chlorophenyl)-6-(5-(1-(methylamino)-2-(methylsulfinyl)ethyl)furan-2-yl)quinazolin-4-amine; (compound 80)
(R)—N-(4-(3-fluorobenzyl-oxy)-3-chlorophenyl)-6-(5-(1-(ethylamino)-2-(methylsulfinyl)ethyl)furan-2-yl)quinazolin-4-amine; (compound 81)
(R)—N-(4-(3-fluorobenzyl-oxy)-3-chlorophenyl)-6-(5-(1-(propylamino)-2-(methylsulfinyl)ethyl)furan-2-yl)quinazolin-4-amine; (compound 82)
(R)—N-(4-(3-fluorobenzyl-oxy)-3-chlorophenyl)-6-(5-(1-(cyclopropyl-methyl-amino)-2-(methylsulfinyl)ethyl)furan-2-yl)quinazolin-4-amine; (compound 83)
(R)—N-(4-(3-fluorobenzyl-oxy)-3-chlorophenyl)-6-(5-(1-(N,N-dimethyl-amino)-2-(methylsulfinyl)ethyl)furan-2-yl)quinazolin-4-amine; (compound 84)
(R)—N-(4-(3-fluorobenzyl-oxy)-3-chlorophenyl)-6-(5-(1-(N,N-diethylamino)-2-(methylsulfinyl)ethyl)furan-2-yl)quinazolin-4-amine; (compound 85)
(R)—N-(4-(3-fluorobenzyl-oxy)-3-chlorophenyl)-6-(5-(1-(N,N-dipropyl-amino)-2-(methylsulfinyl)ethyl)furan-2-yl)quinazolin-4-amine; (compound 86)
(R)—N-(4-(3-fluorobenzyl-oxy)-3-chlorophenyl)-6-(5-(1-(N-methyl, N-ethyl-amino)-2-(methylsulfinyl)ethyl)furan-2-yl)quinazolin-4-amine; (compound 87)
(R)—N-(4-(3-fluorobenzyl-oxy)-3-chlorophenyl)-6-(5-(1-(allyl-amino)-2-(methylsulfinyl)ethyl)furan-2-yl)quinazolin-4-amine; (compound 88)
(R)—N-(4-(3-fluorobenzyl-oxy)-3-chlorophenyl)-6-(5-(1-(propargylamino)-2-(methylsulfinyl)ethyl)furan-2-yl)quinazolin-4-amine; (compound 89)
(S)—N-(4-(3-fluorobenzyl-oxy)-3-chlorophenyl)-6-(5-(1-(amino)-2-(methylthio)ethyl)furan-2-yl)quinazolin-4-amine; (compound 1)
(S)—N-(4-(3-fluorobenzyl-oxy)-3-chlorophenyl)-6-(5-(1-(methylamino)-2-(methylthio)ethyl)furan-2-yl)quinazolin-4-amine; (compound 2)
(S)—N-(4-(3-fluorobenzyl-oxy)-3-chlorophenyl)-6-(5-(1-(ethylamino)-2-(methyl-thio)ethyl)furan-2-yl)quinazolin-4-amine; (compound 3)
(S)—N-(4-(3-fluorobenzyl-oxy)-3-chlorophenyl)-6-(5-(1-(propylamino)-2-(methylthio)ethyl)furan-2-yl)quinazolin-4-amine; (compound 4)
(S)—N-(4-(3-fluorobenzyl-oxy)-3-chlorophenyl)-6-(5-(1-(cyclopropyl-methyl-amino)-2-(methylthio)ethyl)furan-2-yl)quinazolin-4-amine; (compound 5)
(S)—N-(4-(3-fluorobenzyl-oxy)-3-chlorophenyl)-6-(5-(1-(N,N-dimethyl-amino)-2-(methylthio)ethyl)furan-2-yl)quinazolin-4-amine; (compound 6)
(S)—N-(4-(3-fluorobenzyl-oxy)-3-chlorophenyl)-6-(5-(1-(N,N-diethylamino)-2-(methylthio)ethyl)furan-2-yl)quinazolin-4-amine; (compound 7)
(S)—N-(4-(3-fluorobenzyl-oxy)-3-chlorophenyl)-6-(5-(1-(N,N-dipropyl-amino)-2-(methylthio)ethyl)furan-2-yl)quinazolin-4-amine; (compound 8)
(S)—N-(4-(3-fluorobenzyl-oxy)-3-chlorophenyl)-6-(5-(1-(N-methyl, N-ethyl-amino)-2-(methylthio)ethyl)furan-2-yl)quinazolin-4-amine; (compound 9)
(S)—N-(4-(3-fluorobenzyl-oxy)-3-chlorophenyl)-6-(5-(1-(allyl-amino)-2-(methylthio)ethyl)furan-2-yl)quinazolin-4-amine; (compound 10)
(S)—N-(4-(3-fluorobenzyl-oxy)-3-chlorophenyl)-6-(5-(1-(propargylamino)-2-(methylthio)ethyl)furan-2-yl)quinazolin-4-amine; (compound 11)
(S)—N-(1-(3-fluorobenzyl)-1H-indazol-5-yl)-6-(5-(1-(methylamino)-2-(methylthio)ethyl)furan-2-yl)quinazolin-4-amine; (compound 28)
(S)—N-(1-(3-fluorobenzyl)-1H-indazol-5-yl)-6-(5-(1-(ethylamino)-2-(methylthio)ethyl)furan-2-yl)quinazolin-4-amine; (compound 29)
(S)—N-(1-(3-fluorobenzyl)-1H-indazol-5-yl)-6-(5-(1-(propyl-amino)-2-(methylthio)ethyl)furan-2-yl)quinazolin-4-amine; (compound 30)
(S)—N-(1-(3-fluorobenzyl)-1H-indazol-5-yl)-6-(5-(1-(N,N-dimethyl-amino)-2-(methylthio)ethyl)furan-2-yl)quinazolin-4-amine; (compound 32)
(S)—N-(1-(3-fluorobenzyl)-1H-indazol-5-yl)-6-(5-(1-(cyclopropyl-methyl-amino)-2-(methylthio)ethyl)furan-2-yl)quinazolin-4-amine; (compound 31)
(S)—N-(1-(3-fluorobenzyl)-1H-indazol-5-yl)-6-(5-(1-(N-methyl, N-ethyl-amino)-2-(methylthio)ethyl)furan-2-yl)quinazolin-4-amine; (compound 35)
(S)—N-(1-(3-fluorobenzyl)-1H-indazol-5-yl)-6-(5-(1-(allyl-amino)-2-(methylthio)ethyl)furan-2-yl)quinazolin-4-amine; (compound 36)
(S)—N-(1-(3-fluorobenzyl)-1H-indazol-5-yl)-6-(5-(1-(propargylamino)-2-(methylthio)ethyl)furan-2-yl)quinazolin-4-amine; (compound 37);
(S)—N-(4-(3-fluorobenzyl-oxy)-3-chlorophenyl)-6-(5-(1-(methylamino)-2-(methylsulfinyl)ethyl)furan-2-yl)quinazolin-4-amine (compound 67);
(S)—N-(4-(3-fluorobenzyl-oxy)-3-chlorophenyl)-6-(5-(1-(ethylamino)-2-(methylsulfinyl)ethyl)furan-2-yl)quinazolin-4-amine (compound 68);
(S)—N-(4-(3-fluorobenzyl-oxy)-3-chlorophenyl)-6-(5-(1-(propylamino)-2-(methylsulfinyl)ethyl)furan-2-yl)quinazolin-4-amine (compound 69);
(S)—N-(4-(3-fluorobenzyl-oxy)-3-chlorophenyl)-6-(5-(1-(cyclopropyl-methyl-amino)-2-(methylsulfinyl)ethyl)furan-2-yl)quinazolin-4-amine (compound 70);
(S)—N-(4-(3-fluorobenzyl-oxy)-3-chlorophenyl)-6-(5-(1-(N,N-dimethyl-amino)-2-(Methylsulfinyl)ethyl)furan-2-yl)quinazolin-4-amine (compound 71);
(S)—N-(4-(3-fluorobenzyl-oxy)-3-chlorophenyl)-6-(5-(1-(N,N-diethyl-amino)-2-(Methylsulfinyl)ethyl)furan-2-yl)quinazolin-4-amine (compound 72);
(S)—N-(4-(3-fluorobenzyl-oxy)-3-chlorophenyl)-6-(5-(1-(N,N-dipropyl-amino)-2-(methylsulfinyl)ethyl)furan-2-yl)quinazolin-4-amine (compound 73);
(S)—N-(4-(3-fluorobenzyl-oxy)-3-chlorophenyl)-6-(5-(1-(N-methyl, N-ethyl-amino)-2-(Methylsulfinyl)ethyl)furan-2-yl)quinazolin-4-amine (compound 74);
(S)—N-(4-(3-fluorobenzyl-oxy)-3-chlorophenyl)-6-(5-(1-(allyl-amino)-2-(methylsulfinyl)ethyl)furan-2-yl)quinazolin-4-amine (compound 75); and
(S)—N-(4-(3-fluorobenzyl-oxy)-3-chlorophenyl)-6-(5-(1-(propargyl-amino)-2-(methylsulfinyl)ethyl)furan-2-yl)quinazolin-4-amine (compound 76).

The invention also aims to provide a pharmaceutical composition comprising the above-mentioned therapeutical compound of general formula (I) or pharmaceutically acceptable salts in an effective amount thereof and pharmaceutically acceptable carriers.

The invention also aims to provide the use of the compounds of general formula (I) or pharmaceutically acceptable salts thereof in the preparation of a medicament for the treatment of diseases associated with regulating c-erbB-2 and/or EGF-R protein tyrosine kinase activity.

The invention also aims to provide the use of the pharmaceutical composition comprising the compounds of general formula (I) or pharmaceutically acceptable salts thereof in the preparation of a medicament for the treatment of diseases associated with regulating c-erbB-2 and/or EGF-R protein tyrosine kinase activity.

The invention also aims to provide the use of the compounds of general formula (I) or pharmaceutically acceptable salts thereof in the preparation of a medicament for the treatment of cancer and malignant tumors.

The invention also aims to provide the use of the compounds of general formula (I) or pharmaceutically acceptable salts thereof in the preparation of a medicament for the treatment of psoriasis.

The invention also aims to provide the use of the pharmaceutical composition comprising the compounds of general formula (I) or pharmaceutically acceptable salts thereof in the preparation of a medicament for the treatment of cancer and malignant tumors.

The invention also aims to provide the use of the pharmaceutical composition comprising the compounds of general formula (I) or pharmaceutically acceptable salts thereof in the preparation of a medicament for the treatment of psoriasis.

The pharmaceutical preparations in the present invention can occur as a unit dose, with each unit dose containing a predetermined quantity of active ingredient. Such unit dose may contain such as 0.5 mg-1 g. The specific dosage depends on the diseases, routes of administration and the patient's age, weight, condition and other factors.

The pharmaceutical preparations can be administrated by any suitable methods, such as oral, rectal, nasal, local or parenteral (including subcutaneous, intramuscular, intravenous or transdermal) administration etc. The above pharmaceutical preparations can be prepared by any methods known in the pharmaceutical field, such as by mixing active ingredients with a carrier or an excipient.

The compounds or pharmaceutically acceptable salts thereof of the invention can be administrated alone or in combination with other therapeutic agents for treatment of the above diseases. Administration in combination with other chemotherapeutic agents, hormones or antibody drugs should be considered, especially in anti-tumor therapy.

### EMBODIMENTS

To describe the present invention in more detail, the following examples are provided. However, the scope of the present invention is not limited to these.

The enantiomer excesses (e.e) in the following embodiment refer to the relative amount of each enantiomer. The value is defined as the difference of the relative percentage of two enantiomers. For example, when the percentage of (R) enantiomer is 90%, and the percentage of (S) enantiomer is 10%, then the value of e.e is 80%.

A chiral high performance liquid chromatography (HPLC) was used to measure the enantiomer of each compound, the method is as below:

Column: Daicel AD;

Mobile phase: n-hexane-ethanol-diethylamine (50:50:0.1).

### Example 1

### Preparation of N-(4-(3-fluorobenzyloxy)-3-chlorophenyl)-6-iodine-quinazolin-4-amine

6-Iodine-3H-quinazolin-4-ketone (100 g) was added into a 2000 mL flask, dissolved in a mixed solvent of thionyl chloride (1000 mL) and N,N-dimethylformamide (20 mL), heated to reflux until the reaction solution is clear and transparent. After thionyl chloride was removed, anhydrous toluene was added to the residues and removed under reduced pressure, and the process of the adding and removing of toluene was repeated again to removed the remained thionyl chloride residues.

The intermediate was dissolved in isopropyl alcohol (2000 mL), 3-chloro-4-(3-fluoro-benzyloxy)-aniline hydrochloride was added, and anhydrous K₂CO₃ (150 g) was added with mechanical stirring before the mixture was heated to reflux over night. The reaction solution was cooled to room temperature overnight, the precipitation was filtered and washed with water for multi-times until the pH of washing solution reached neutral. After drying under vacuum, 95 g of the title product was collected in a pale white solid.

m/z M⁺1⁺: 506

### Example 2

### Preparation of N-(1-(3-fluorobenzyl)-1H-indazol-5-yl)-6-iodoquinazolin-4-amine

The process is the same as that in example 1, except that 3-chloro-4-(3-fluoro-benzyloxy)-aniline hydrochloride was replaced by 1-(3-fluorobenzyl)-1H-indazol-5-amine hydrochloride.

m/z (M+1)⁺: 496.

### Example 3

### Preparation of 5-(4-(4-(3-fluorobenzyl-oxy)-3-chlorophenyl-amino)quinazolin-6-yl)furan-2-aldehyde

Product (50 g) of example 1,5-boric acid-2-furfural (21 g), Pd(PPh₃)₂Cl₂(6.2 g) triethylamine (62 mL), and methyl alcohol (1000 mL) were added into a reaction flask. The mixture was refluxed for 2 hours. After the reaction solution was cooled to room temperature, the precipitation was filtered and washed by a small amount of methanol, then dried at 50° C. to obtain the 40 g the subject product in a yellow solid.

m/z (M+1)⁺: 473.

### Example 4

### Preparation of 5-(4-(1-(3-fluorobenzyl)-1H-indazol-5-yl-amino) quinazolin-6-yl)furan-2-aldehyde

The process is the same as in example 3, except that the raw material, compound of example 1 was replaced by compound of example 2.

m/z (M+1)⁺: 464.

### Example 5

### Preparation of 2-(4-(4-(3-fluorobenzyl-oxy)-3-chlorophenyl-amino)quinazolin-6-yl)thiazole-5-aldehyde

Product (50 g) of example 1,2-boric acid-5-thiazole aldehyde (21 g), Pd(PPh₃)₂Cl₂(6.2 g), triethylamine (62 mL), and methyl alcohol (1000 mL) were added into a reaction flask. The mixture was refluxed for 2 hours. After the reaction solution was cooled to room temperature, the precipitation was filtered and washed by a small amount of methanol, then dried at 50° C. to obtain 30 g title product.

m/z (M+1)⁺: 490.

### Example 6

### Preparation of 2-(4-(1-(3-fluorobenzyl)-1H-indazol-5-amino)quinazolin-6-yl)thiazole-5-aldehyde

The process is the same as the process in example 5, except that the raw material, compound of example 1 was replaced by compound of example 2.

m/z (M+1)⁺: 480.

### Embodiment 1

### Preparation of (S)—N-((5-(4-(4-(3-fluorobenzyl-oxy)-3-chlorophenyl-amino)quinazolin-6-yl)furan-2-yl)methylenyl)-2-methylpropane-2-sulfinamide

Product of example 3 (47.3 g, 0.1 mol), (S)-(−)-2-methyl-2-propanesulfinamide (14.5 g, 0.12 mol), titanium (IV) isopropoxide (tetra isopropyl titanate) (85 g, 0.3 mol) and anhydrous THF (1000 mL) were added into a reaction flask and reacted at room temperature over night. Then water (50 mL) and ethyl acetate (500 mL) were added with stirring for 10 minutes, followed by a filtration and the precipitation was washed with THF for three times. The combined filtrates were dried with anhydrous magnesium sulfate, filtered and concentrated under reduced pressure to dry to obtain 50 g solid title compound.

m/z (M+1)⁺: 577.

### Embodiment 2

### Preparation of (R)—N-((5-(4-(4-(3-fluorobenzyl-oxy)-3-chlorophenyl-amino)quinazolin-6-yl)furan-2-yl)methylenyl)-2-methylpropane-2-sulfinamide

The process is the same as the process in embodiment 1, except that the raw material, (S)-(−)-2-methyl-2-propanesulfinamide was replaced by (R)-(+)-2-methyl-2-propanesulfinamide.

m/z (M+1)⁺: 577.

### Embodiment 3

### Preparation of (S)—N-((2-(4-(4-(3-fluorobenzyl-oxy)-3-chlorophenyl-amino)quinazolin-6-yl)thiazole-5-yl)methylenyl)-2-methylpropane-2-sulfinamide

The process is the same as the process in embodiment 1, except that the raw material compound of example 3 was replaced by compound of example 5.

m/z (M+1)⁺: 594.

### Embodiment 4

### Preparation of (R)—N-((2-(4-(4-(3-fluorobenzyl-oxy)-3-chlorophenyl-amino)quinazolin-6-yl)thiazole-5-yl)methylenyl)-2-methylpropane-2-sulfinamide

The process is the same as the process in embodiment 3, except that the raw material (S)-(−)-2-methyl-2-propanesulfinamide was replaced by (R)-(+)-2-methyl-2-propanesulfinamide.

m/z (M+1)⁺: 594

### Embodiment 5

### Preparation of (S)—N-((5-(4-(1-(3-fluorobenzyl)-1H-indazol-5-amino)quinazolin-6-yl)furan-2-yl)methylenyl)-2-methylpropane-2-sulfinamide

The process is the same as the process in embodiment 1, except that the raw material compound of example 3 was replaced by compound of example 4.

m/z (M+1)⁺: 567.

### Embodiment 6

### Preparation of (R)—N-((5-(4-(1-(3-fluorobenzyl)-1H-indazol-5-amino)quinazolin-6-yl)furan-2-yl)methylenyl)-2-methylpropane-2-sulfinamide

The process is the same as the process in embodiment 2, except that the raw material compound of example 3 was replaced by compound of example 4.

m/z (M+1)⁺: 567.

### Embodiment 7

### Preparation of (S)—N-((2-(4-(1-(3-fluorobenzyl)-1H-indazol-5-amino)quinazolin-6-yl)thiazole-5-yl)methylenyl)-2-methylpropane-2-sulfinamide

The process is the same as the process in embodiment 1, except that the raw material compound of example 3 was replaced by compound of example 6.

m/z (M+1)⁺: 584

### Embodiment 8

### Preparation of (R)—N-((2-(4-(1-(3-fluorobenzyl)-1H-indazol-5-amino)quinazolin-6-yl)thiazole-5-yl)methylenyl)-2-methylpropane-2-sulfinamide

The process is the same as the process in embodiment 1, except that the raw material compound of example 3 was replaced by compound of example 6. m/z (M+1)⁺: 584.

### Embodiment 9

### Preparation of (S)—N-(4-(3-fluorobenzyl-oxy)-3-chlorophenyl)-6-(5-(1-(amino)-2-(methylthio)ethyl)furan-2-yl)quinazolin-4-amine

Methylthio-methyl magnesium chloride/THF solution (0.3 mol) was added into a reaction flask and the reaction solution was cooled below −80° C. Then, the solution of product of embodiment 1 (57.6 g, 0.1 mol) in anhydrous THF (200 mL) was added to the flask rapidly at −80° C. and stirring for 10 minutes at the same temperature. Saturated saline (3000 mL) was decanted into the reaction solution, and ethyl acetate (2000 mL) was added to extract the product. The organic layer was washed with saturated saline (2000 mL), and dried with anhydrous magnesium sulfate followed by a filtration to obtain 50 g yellow solid by concentrating the filtrate under reduced pressure.

The yellow solid from the above step was dissolved in THF (1000 mL) and pH of the solution was adjusted to 1 with HCl-ethanol and the solution was stirred for 2 hours at room temperature. After pH of the solution was adjusted to 9 with ammonia, the saturated saline (2000 mL) and ethyl acetate (1500 mL) were added to the solution. The organic layer was separated and dried with anhydrous magnesium sulfate followed by a filtration and concentrated under reduced pressure. The residue was purified by silica-gel column chromatography (Eluent: ethyl acetate-ethyl acetate/THF=10/1) and the fraction in need was collected, concentrated to obtain 30 g title product numbered as Compound 1. m/z (M+1)⁺: 535

### Embodiment 10

### Preparation of (S)—N-(4-(3-fluorobenzyl-oxy)-3-chlorophenyl)-6-(5-(1-(methylamino)-2-(methylthio)ethyl)furan-2-yl)quinazolin-4-amine

Method A: the product (2.0 g) obtained in embodiment 9, iodomethane (0.5 g) and triethylamine (0.7 g) were dissolved in THF (150 mL), heated to reflux for 2 hours, then cooled to room temperature. To the reaction solution, saturated saline and ethyl acetate were added. The separated organic layer was washed with saturated saline twice, dried with anhydrous magnesium sulfate. After the filtration and concentration under reduced pressure, the residue was purified by silica-gel column chromatography (Chloroform/methanol=100:1), to obtain the title product (1.5 g) numbered as Compound 2.

Method B: the product (2.0 g) from embodiment 9 was dissolved in DMSO (50 mL), then formaldehyde (6 mL), formic acid (3 mL) were added and the solution was stirred over night at room temperature. The reaction solution was mixed with ice water (500 mL) and solid was filtered and collected. After dissolving with THF, the solution of the solid collection was purified by silica-gel column chromatography to obtain 4.2 g title product numbered as Compound 2. m/z (M+1)⁺: 549.

According to the preparation methods in embodiment 10, the compound obtained in embodiment 9, as the starting material, was used to react with reagents to prepare the following compounds:

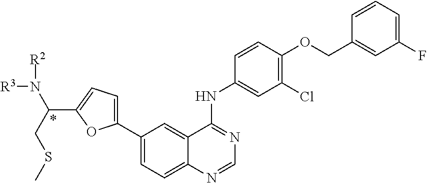

| | | | | m/z |
|---|---|---|---|---|
| | | | | (M + |
| Number | Reagent | R² = | R³ = | 1)⁺ |
| Com- | Ethyl iodide | H | —CH₂CH₃ | 563 |
| pound 3 | | | | |
| Com- | Iodine | H | —CH₂CH₂CH₂ | 577 |
| pound 4 | propane | | | |
| Com- pound 5 | Cyclopropyl- methyl- bromide | H | 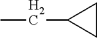 | 589 |
| Com- | Iodomethane | —CH₃ | —CH₃ | 563 |
| pound 6 | | | | |
| Com- | Ethyl iodide | —CH₂CH₃ | —CH₂CH₃ | 591 |
| pound 7 | | | | |
| Com- | CH₃CH₂CH₂I | —CH₂CH₂CH₃ | —CH₂CH₂CH₃ | 619 |
| pound 8 | | | | |
| Com- | CH₃CH₂I/ | —CH₃ | —CH₂CH₃ | 577 |
| pound 9 | CH₃I | | | |
| Com- pound 10 | Allyl bromide | H | 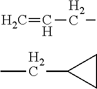 | 575 |
| Com- pound 11 | Propargyl bromide | H | 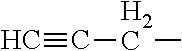 | 573 |
| Com- | CH₃OBr | H | —OCH₃ | 565 |
| pound 12 | | | | |
| Com- | CH₃OBr/ | —CH₃ | —OCH₃ | 579 |
| pound 13 | CH₃I | | | |

Remark: Carbon atom with * is (S)-configuration

### Embodiment 11

### Preparation of (S)—N-(4-(3-fluorobenzyl-oxy)-3-chlorophenyl)-6-(5-(1-(amino)-2-(methylthio)ethyl)thiazole-2-yl)quinazolin-4-amine

The process is the same as the process in embodiment 9, except that the raw material compound of embodiment 1 was replaced by compound of embodiment 3.

m/z (M+1)⁺: 552

According to the preparation methods in embodiment 10, the compound in embodiment 11, as the starting material, was used to react with reagents to prepare the following compounds:

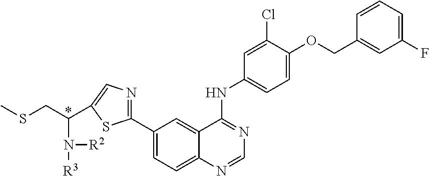

| | | | | m/z |
|---|---|---|---|---|
| | | | | (M + |
| Number | Regent | R² = | R³ = | 1)⁺ |
| Com- | CH₃I | H | —CH₃ | 566 |
| pound 15 | | | | |
| Com- | CH₃CH₂—I | H | —CH₂CH₃ | 580 |
| pound 16 | | | | |
| Com- | CH₃CH₂CH₂—I | H | —CH₂CH₂CH₃ | 594 |
| pound 17 | | | | |
| Com- pound 18 | Cyclopropyl- methylbromide | H | 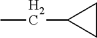 | 606 |
| Com- | CH₃I | —CH₃ | —CH₃ | 580 |
| pound 19 | | | | |
| Com- | CH₃CH₂I | —CH₂CH₃ | —CH₂CH₃ | 608 |
| pound 20 | | | | |
| Com- | CH₃CH₂CH₂I | —CH₂CH₂CH₃ | —CH₂CH₂CH₃ | 636 |
| pound 21 | | | | |
| Com- | CH₃CH₂I/CH₃I | —CH₃ | —CH₂CH₃ | 594 |
| pound 22 | | | | |
| Com- pound 23 | Allyl bromide | H | 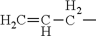 | 592 |
| Com- pound 24 | Propargyl bromide | H | 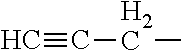 | 590 |
| Com- | CH₃OBr | H | —OCH₃ | 582 |
| pound 25 | | | | |
| Com- | CH₃OBr/CH₃I | —CH₃ | —OCH₃ | 596 |
| pound 26 | | | | |

Remark: Carbon atom with * is (S)-configuration

### Embodiment 12

### Preparation of (S)—N-(1-(3-fluorobenzyl)-1H-indazol-5-yl)-6-(5-(1-(amino)-2-(methylthio)ethyl)furan-2-yl)quinazolin-4-amine

The process is the same as the process in embodiment 9, except that the raw material compound of embodiment 1 was replaced by compound of embodiment 5 to obtain the titled compound (Compound 27).

m/z (M+1)⁺:525

According to the preparation methods in embodiment 10, the compound in embodiment 12, as the starting material, was used to react with reagents to prepare the following compounds:

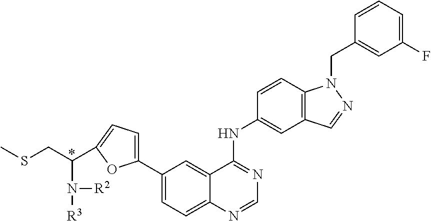

| | | | | m/z |
|---|---|---|---|---|
| | | | | (M + |
| Number | Reagent | R² = | R³ = | 1)⁺ |
| Com- | CH₃I | H | —CH₃ | 539 |
| pound 28 | | | | |
| Com- | CH₃CH₂—I | H | —CH₂CH₃ | 553 |
| pound 29 | | | | |
| Com- | CH₃CH₂CH₂—I | H | —CH₂CH₂CH₃ | 567 |
| pound 30 | | | | |
| Com- pound 31 | Cyclo- propylmethyl- bromide | H | 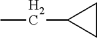 | 579 |
| Com- | CH₃I | —CH₃ | —CH₃ | 553 |
| pound 32 | | | | |
| Com- | CH₃CH₂I | —CH₂CH₃ | —CH₂CH₃ | 581 |
| pound 33 | | | | |
| Com- | CH₃CH₂CH₂I | —CH₂CH₂CH₃ | —CH₂CH₂CH₃ | 609 |
| pound 34 | | | | |
| Com- | CH₃CH₂I/CH₃I | —CH₃ | —CH₂CH₃ | 567 |
| pound 35 | | | | |
| Com- pound 36 | Allyl bromide | H | 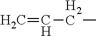 | 565 |
| Com- pound 37 | Propargyl bromide | H | 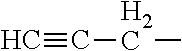 | 563 |
| Com- | CH₃OBr | H | —OCH₃ | 555 |
| pound 38 | | | | |
| Com- | CH₃OBr/CH₃I | —CH₃ | —OCH₃ | 569 |
| pound 39 | | | | |

Remark: Carbon atom with * is (S)-configuration

### Embodiment 13

### Preparation of (R)—N-(4-(3-fluorobenzyl-oxy)-3-chlorophenyl)-6-(5-(1-(amino)-2-(methylthio)ethyl)furan-2-yl)quinazolin-4-amine

The process is the same as the process in embodiment 9, except that the raw material compound of embodiment 1 was replaced by compound of embodiment 2 to obtain the titled compound numbered as Compound 40.

m/z (M+1)⁺: 535

According to the preparation methods in embodiment 10, the compound obtained in embodiment 13, as the starting material, was used to react with reagents to prepare the following compounds:

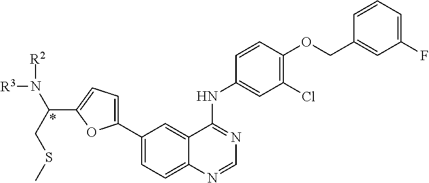

| | | | | m/z |
|---|---|---|---|---|
| | | | | (M + |
| Number | Reagent | R² = | R³ = | 1)⁺ |
| Com- | Iodomethane | H | —CH₃ | 549 |
| pound 41 | | | | |
| Com- | Ethyl iodide | H | —CH₂CH₃ | 563 |
| pound 42 | | | | |
| Com- | Iodine | H | —CH₂CH₂CH₃ | 577 |
| pound 43 | propane | | | |
| Com- pound 44 | Cyclopropyl- methyl- bromide | H | 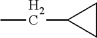 | 589 |
| Com- | Iodomethane | —CH₃ | —CH₃ | 563 |
| pound 45 | | | | |
| Com- | Ethyl iodide | —CH₂CH₃ | —CH₂CH₃ | 591 |
| pound 46 | | | | |
| Com- | CH₃CH₂CH₂I | —CH₂CH₂CH₃ | —CH₂CH₂CH₃ | 619 |
| pound 47 | | | | |
| Com- | CH₃CH₂I/ | —CH₃ | —CH₂CH₃ | 577 |
| pound 48 | CH₃I | | | |
| Com- pound 49 | Allyl bromide | H | 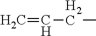 | 575 |
| Com- pound 50 | Propargyl bromide | H | 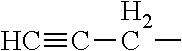 | 573 |
| Com- | CH₃OBr | H | —OCH₃ | 565 |
| pound 51 | | | | |
| Com- | CH₃OBr/ | —CH₃ | —OCH₃ | 579 |
| pound 52 | CH₃I | | | |

Remark: Carbon atom with * is (R)-configuration

### Embodiment 14

### Preparation of (R)—N-(4-(3-fluorobenzyl-oxy)-3-chlorophenyl)-6-(5-(1-(amino)-2-(methylthio)ethyl)thiazole-2-yl)quinazolin-4-amine

The process is the same as that in embodiment 9, except that the raw material compound of embodiment 1 was replace by compound of embodiment 4 to obtain the compound (Compound 53).

m/z (M+1)⁺: 552

According to the preparation methods in embodiment 10, in the compound in embodiment 14, as the starting material, was used to react with reagents to prepare the following compounds:

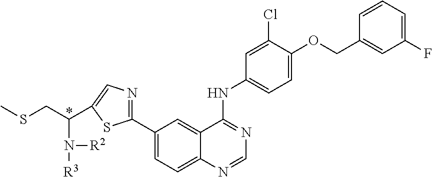

| | | | | m/z |
|---|---|---|---|---|
| | | | | (M + |
| Number | Reagent | R² = | R³ = | 1)⁺ |
| Com- | CH₃I | H | —CH₃ | 566 |
| pound 54 | | | | |
| Com- | CH₃CH₂—I | H | —CH₂CH₃ | 580 |
| pound 55 | | | | |
| Com- | CH₃CH₂CH₂—I | H | —CH₂CH₂CH₃ | 594 |
| pound 56 | | | | |
| Com- pound 57 | Cyclopropyl- methylbromide | H | 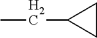 | 606 |
| Com- | CH₃I | —CH₃ | —CH₃ | 580 |
| pound 58 | | | | |
| Com- | CH₃CH₂I | —CH₂CH₃ | —CH₂CH₃ | 608 |
| pound 59 | | | | |
| Com- | CH₃CH₂CH₂I | —CH₂CH₂CH₃ | —CH₂CH₂CH₃ | 636 |
| pound 60 | | | | |
| Com- | CH₃CH₂I/CH₃I | —CH₃ | —CH₂CH₃ | 594 |
| pound 61 | | | | |
| Com- pound 62 | Allyl bromide | H | 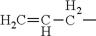 | 592 |
| Com- pound 63 | Propargyl bromide | H | 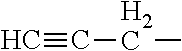 | 590 |
| Com- | CH₃OBr | H | —OCH₃ | 582 |
| pound 64 | | | | |
| Com- | CH₃OBr/CH₃I | —CH₃ | —OCH₃ | 596 |
| pound 65 | | | | |

Remark: Carbon atom with * is (R)-configuration

### Embodiment 15

### Preparation of (S)—N-(4-(3-fluorobenzyl-oxy)-3-chlorophenyl)-6-(5-(1-(amino)-2-(methylsulfinyl)ethyl)furan-2-yl)quinazolin-4-amine

Under the protection of N₂, DMSO (0.4 mol) was dissolved in anhydrous THF (2000 mL) and cooled to −20° C., then n-BuLi (0.3 mol) was added into the above solution dropwisely and stirred for 10 minutes at the same temperature. The reaction solution was cooled below −80° C., then the solution of product of embodiment 1 (57.6 g, 0.1 mol) and anhydrous THF (200 mL) were added rapidly under −80° C. and stirred for 10 minutes. The reaction solution was decanted into saturated saline (3000 mL) and ethyl acetate (2000 mL) was added to extract the product. The organic layer was washed with saturated saline (2000 mL), and dried with anhydrous magnesium sulfate. After filtration and concentration under reduced pressure, 42 g yellow solid was obtained.

The yellow solid obtained in the last step was dissolved in THF (1000 mL) and pH of the solution was adjusted to 1 with HCl-ethanol and stirred for 2 hours at room temperature. To the solution, strong ammonia was added to adjust the pH to 9 and saturated saline (2000 mL) and ethyl acetate (1500 mL) were added to extract the product. The organic layer was dried with anhydrous magnesium sulfate. After filtration and concentrate of the filtrate under reduced pressure, the remainder residue was purified by silica-gel column chromatography (Eluent: ethyl acetate-ethyl acetate/THF=5/1) and the fraction in need was collected and concentrated to obtain 20 g title product (Compound 66).

m/z (M+1)⁺: 551

### Embodiment 16

### Preparation of (S)—N-(4-(3-fluorobenzyl-oxy)-3-chlorophenyl)-6-(5-(1-(methylamino)-2-(methylsulfinyl)ethyl)furan-2-yl)quinazolin-4-amine

The process is the same as the process in embodiment 10, except that the raw material compound of embodiment 7 was replaced by compound of embodiment 15, and the title compound was numbered as Compound 67.

m/z (M+1)⁺: 566

According to the preparation methods in embodiment 10, the compound obtained in embodiment 15, as the starting material, was used to react with reagents to prepare the following compounds:

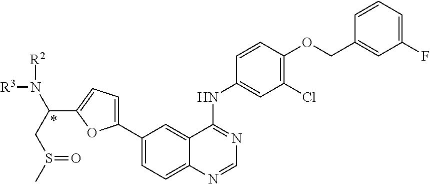

| | | | | m/z |
|---|---|---|---|---|
| | | | | (M + |
| Number | Reagent | R² = | R³ = | 1)⁺ |
| Com- | Ethyl iodide | H | —CH₂CH₃ | 579 |
| pound 68 | | | | |
| Com- | Iodine | H | —CH₂CH₂CH₃ | 593 |
| pound 69 | propane | | | |
| Com- pound 70 | Cyclopropyl- methylbromide | H | 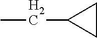 | 605 |
| Com- | CH3I | —CH₃ | —CH₃ | 579 |
| pound 71 | | | | |
| Com- | CH₃CH₂I | —CH₂CH₃ | —CH₂CH₃ | 607 |
| pound 72 | | | | |
| Com- | CH₃CH₂CH₂I | —CH₂CH₂CH₃ | —CH₂CH₂CH₃ | 635 |
| pound 73 | | | | |
| Com- | CH₃CH₂I/CH₃I | —CH₃ | —CH₂CH₃ | 593 |
| pound 74 | | | | |
| Com- pound 75 | Allyl bromide | H | 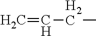 | 591 |
| Com- pound 76 | Propargyl bromide | H | 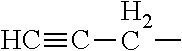 | 589 |
| Com- | CH₃OBr | H | —OCH₃ | 581 |
| pound 77 | | | | |
| Com- | CH₃OBr/CH₃I | —CH₃ | —OCH₃ | 595 |
| pound 78 | | | | |

Remark: Carbon atom with * is (S)-configuration

### Embodiment 17

### Preparation of (R)—N-(4-(3-fluorobenzyl-oxy)-3-chlorophenyl)-6-(5-(1-(amino)-2-(methylsulfinyl)ethyl)furan-2-yl)quinazolin-4-amine

The process is the same as the process in embodiment 15, except that the raw material compound of embodiment 1 was replaced by compound of embodiment 2, and the title compound was numbered as Compound 79. m/z (M+1)⁺: 551

According to the preparation methods in embodiment 10, the compound obtained in embodiment 17, as the starting material, was used to react with reagents to prepare the following compounds:

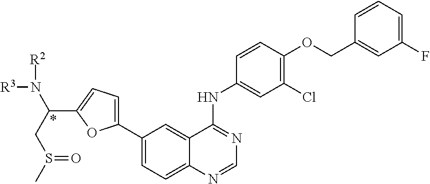

| | | | | m/z |
|---|---|---|---|---|
| | | | | (M + |
| Number | Reagent | R² = | R³ = | 1)⁺ |
| Com- | CH₃I | H | —CH₃ | 565 |
| pound 80 | | | | |
| Com- | CH₃CH₂I | H | —CH₂CH₃ | 579 |
| pound 81 | | | | |
| Com- | CH₃CH₂CH₂I | H | —CH₂CH₂CH₃ | 593 |
| pound 82 | | | | |
| Com- pound 83 | Cyclopropyl- methylbromide | H | 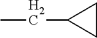 | 605 |
| Com- | CH₃I | —CH₃ | —CH₃ | 579 |
| pound 84 | | | | |
| Com- | CH₃CH₂I | —CH₂CH₃ | —CH₂CH₃ | 607 |
| pound 85 | | | | |
| Com- | CH₃CH₂CH₂I | —CH₂CH₂CH₃ | —CH₂CH₂CH₃ | 635 |
| pound 86 | | | | |
| Com- | CH₃CH₂I/CH₃I | —CH₃ | —CH₂CH₃ | 593 |
| pound 87 | | | | |
| Com- pound 88 | Allyl bromide | H | 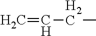 | 591 |
| Com- pound 89 | Propargyl bromide | H | 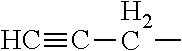 | 589 |
| Com- | CH₃OBr | H | —OCH₃ | 581 |
| pound 90 | | | | |
| Com- | CH₃OBr/CH₃I | —CH₃ | —OCH₃ | 595 |
| pound 91 | | | | |

Remark: Carbon atom with * is (R)-configuration

### Embodiment 18

### Preparation of (S)—N-(4-(3-fluorobenzyl-oxy)-3-chlorophenyl)-6-(5-(1-(amino)-2-(methylsulfonyl)ethyl)furan-2-yl)quinazolin-4-amine

Method A: Under the nitrogen protection, DMSO (37.6 g, 0.4 mol) was dissolved in anhydrous THF (2000 mL) and cooled to −20° C., and then n-BuLi (0.3 mol) was added into the above solution dropwisely and the solution was stirred for 30 minutes at the same temperature. The reaction solution was cooled below −80° C., and the solution of product of embodiment 1 (57.6 g, 0.1 mol) and anhydrous THF (200 mL) was added rapidly under −80° C. and the solution was stirred for 10 minutes at the present temperature. The reaction solution was decanted into saturated saline (3000 mL), and ethyl acetate (2000 mL) was added to extract the mixture; the organic layer was washed with saturated saline (2000 mL) and dried with anhydrous magnesium sulfate. After filtration and concentration under reduced pressure, 42 g yellow solid was obtained.

The yellow solid obtained in the last step was dissolved in THF (1000 mL), pH of the solution was adjusted to 1 with HCl-ethanol and was stirred for 2 h at room temperature. The pH of the solution was adjusted with strong ammonia to 9, saturated saline (2000 mL) and ethyl acetate (1500 mL) were added to extract the mixture. The organic layer was dried with anhydrous magnesium sulfate. After filtration, concentration under reduced pressure, the residue from organic layer was purified by silica-gel column chromatography (Eluent: ethyl acetate-ethyl acetate/THF=5/1), the fraction in need was collected, concentrated to obtain 20 g title product numbered as Compound 92.

Method B: The product obtained in embodiment 9 (50 g) and embodiment 15 (50 g) and mixed solvent of methanol/water (7:3, 1000 mL) were added into a reaction flask. After all products were dissolved in the solvent, potassium peroxymonopersulfate (KHS05) 100 g was added in batches with stirring for 2 hours at room temperature. After filtration and the solid residue was washed with mixed solvent of methanol/water, and pH of the combined filtrate was adjusted to 8 with saturated sodium bicarbonate solution, the solution was concentrated under reduced pressure, and then extracted by ethyl acetate (500 mL×2). The organic layers were combined, and dried with anhydrous sodium sulfate. After filtration and concentration under reduced pressure, the residue was purified by silica-gel column chromatography (Eluent: chloroform/methanol=100:1) to obtain the title product: 40 g, yellow solid. m/z (M+1)⁺: 567; e.e value: 95.2%, [(S):97.6% (R) 2.4%]

According to the preparation methods in embodiment 10, the compound obtained in embodiment 18, as the starting material, was used to react with reagents to prepare the following compounds:

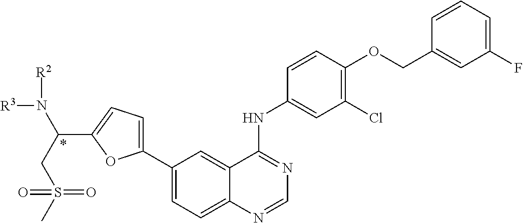

| | | | | m/z | e.e. |
|---|---|---|---|---|---|
| Number | Reagent | R² = | R³ = | (M + 1)⁺ | value % |
| Compound 93 | CH₃I | H | —CH₃ | 581 | 95.2 |
| Compound 94 | CH₃CH₂I | H | —CH₂CH₃ | 595 | 95.3 |
| Compound 95 | CH₃CH₂CH₂I | H | —CH₂CH₂CH₃ | 609 | 95.3 |
| Compound 96 | Cyclopropyl- methylbromide | H | 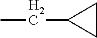 | 621 | 95.2 |
| Compound 97 | CH₃I | —CH₃ | —CH₃ | 595 | 95.2 |
| Compound 98 | CH₃CH₂I | —CH₂CH₃ | —CH₂CH₃ | 623 | 95.2 |
| Compound 99 | CH₃CH₂CH₂I | —CH₂CH₂CH₃ | —CH₂CH₂CH₃ | 651 | 95.2 |
| Compound 100 | CH₃CH₂I/CH₃I | —CH₃ | —CH₂CH₃ | 609 | 95.4 |
| Compound 101 | Allyl bromide | H | 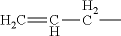 | 607 | 95.2 |
| Compound 102 | Propargyl bromide | H | 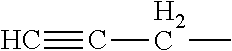 | 605 | 95.2 |
| Compound 103 | CH₃OBr | H | —OCH₃ | 597 | 9⁵.1 |
| Compound 104 | CH₃OBr/CH₃I | —CH₃ | —OCH₃ | 611 | 95.2 |

Remark: Carbon atom with * is (S)-configuration

### Embodiment 19

### Preparation of (R)—N-(4-(3-fluorobenzyl-oxy)-3-chlorophenyl)-6-(5-(1-(amino)-2-(methylsulfonyl)ethyl)furan-2-yl)quinazolin-4-amine

Method A: The process is as same as the method A in embodiment 18, except that the raw material, compound of embodiment 1, was replaced by compound of embodiment 2, and the product was numbered as Compound 105.

m/z (M+1)⁺: 567; e.e value: 95.8%, [(R):97.9% (S): 2.1%]

Method B: The process is the same as the method B in embodiment 18, except that the raw materials, compound of embodiment 9 and embodiment 15, were replaced by compound of embodiment 13 or embodiment 17.

According to the preparation methods in embodiment 10, the compound obtained in embodiment 19, as the starting material, was used to react with reagents to prepare the following compounds:

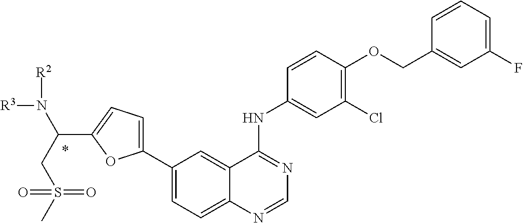

| | | | | m/z | e.e. |
|---|---|---|---|---|---|
| Number | Reagent | R² = | R³ = | (M + 1)⁺ | value % |
| Compound 106 | CH₃I | H | —CH₃ | 581 | 95.9 |
| Compound 107 | CH₃CH₂I | H | —CH₂CH₃ | 595 | 95.8 |
| Compound 108 | CH₃CH₂CH₂I | H | —CH₂CH₂CH₃ | 609 | 95.8 |
| Compound 109 | Cyclopropyl- methylbromide | H | 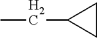 | 621 | 95.9 |
| Compound 110 | CH₃I | —CH₃ | —CH₃ | 595 | 96.0 |
| Compound 111 | CH₃CH₂I | —CH₂CH₃ | —CH₂CH₃ | 623 | 95.8 |
| Compound 112 | CH₃CH₂CH₂I | —CH₂CH₂CH₃ | —CH₂CH₂CH₃ | 651 | 95.8 |
| Compound 113 | CH₃CH₂I/CH₃I | —CH₃ | —CH₂CH₃ | 609 | 95.7 |
| Compound 114 | Allyl bromide | H | 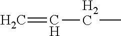 | 607 | 95.8 |
| Compound 115 | Propargyl bromide | H | 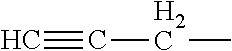 | 605 | 95.9 |
| Compound 116 | CH₃OBr | H | —OCH₃ | 597 | 95.8 |
| Compound 117 | CH₃OBr/CH₃I | —CH3 | —OCH₃ | 611 | 95.8 |

Remark: Carbon atom with * is (R)-configuration

### Embodiment 20

### Preparation of (S)—N-(4-(3-fluorobenzyl-oxy)-3-chlorophenyl)-6-(5-(1-(amino)-2-(methylsulfonyl)ethyl)thiazole-2-yl)quinazolin-4-amine

The process is the same as the process described in the method A in embodiment 18, except that the raw material, compound of embodiment 1 was replaced by compound of embodiment 3, and the product was numbered as Compound 118.

m/z (M+1)⁺: 584; e.e value: 91.4%[(S):95.7%, (R): 4.3%]

According to the preparation methods in embodiment 10, the compound obtained in embodiment 20, as the starting material, was used to react with reagents to prepare the following compounds:

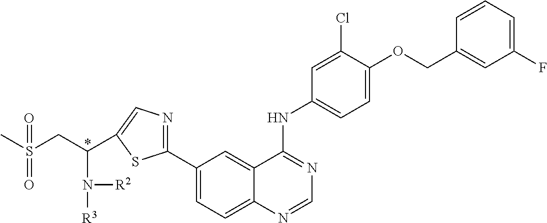

| | | | | m/z | e.e. |
|---|---|---|---|---|---|
| Number | Reagent | R² = | R³ = | (M + 1)⁺ | value % |
| Compound 119 | CH₃I | H | —CH₃ | 598 | 91.5 |
| Compound 120 | CH₃CH₂I | H | —CH₂CH₃ | 612 | 91.7 |
| Compound 121 | CH₃CH₂CH₂I | H | —CH₂CH₂CH₃ | 626 | 91.6 |
| Compound 122 | Cyclopropyl- methylbromide | H | 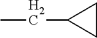 | 638 | 91.5 |
| Compound 123 | CH₃I | —CH₃ | —CH₃ | 612 | 91.4 |
| Compound 124 | CH₃CH₂I | —CH₂CH₃ | —CH₂CH₃ | 640 | 91.8 |
| Compound 125 | CH₃CH₂CH₂I | —CH₂CH₂CH₃ | —CH₂CH₂CH₃ | 668 | 91.5 |
| Compound 126 | CH₃CH₂I/CH₃I | —CH₃ | —CH₂CH₃ | 626 | 91.5 |
| Compound 127 | Allyl bromide | H | 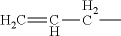 | 624 | 91.6 |
| Compound 128 | Propargyl bromide | H | 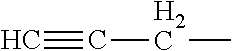 | 622 | 91.7 |
| Compound 129 | CH₃OBr | H | —OCH₃ | 614 | 91.4 |
| Compound 130 | CH₃OBr/CH₃I | —CH₃ | —OCH₃ | 628 | 91.2 |

Remark: Carbon atom with * is (S)-configuration

### Embodiment 21

### Preparation of (R)—N-(4-(3-fluorobenzyl-oxy)-3-chlorophenyl)-6-(5-(1-(amino)-2-(methylsulfonyl)ethyl)thiazole-2-yl)quinazolin-4-amine

m/z (M+1)⁺: 584; e.e 92.2%; [(R):96.1%, (S): 3.9%]

The process is the same as the process in the method A in embodiment 18, except that the raw material, compound of embodiment 1 was replaced by compound of embodiment 4, and the product was numbered as Compound 131.

m/z (M+1)⁺: 584; e.e value: 92.2%; [(R):96.1%, (S): 3.9%]

According to the preparation method in embodiment 10, the compound obtained in embodiment 21, as the starting material, was used to react with reagents to prepare the following compound:

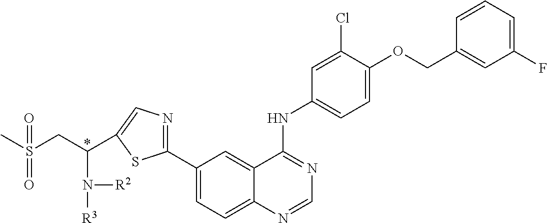

| | | | | m/z | e.e. |
|---|---|---|---|---|---|
| Number | Reagent | R² = | R³ = | (M + 1)⁺ | value % |
| Compound 132 | CH₃I | H | —CH₃ | 598 | 92.3 |
| Compound 133 | CH₃CH₂I | H | —CH₂CH₃ | 612 | 92.0 |
| Compound 134 | CH₃CH₂CH₂I | H | —CH₂CH₂CH₃ | 626 | 92.2 |
| Compound 135 | Cyclopropyl- methylbromide | H | 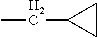 | 638 | 92.1 |
| Compound 136 | CH₃I | —CH₃ | —CH₃ | 612 | 92.2 |
| Compound 137 | CH₃CH₂I | —CH₂CH₃ | —CH₂CH₃ | 640 | 92.3 |
| Compound 138 | CH₃CH₂CH₂I | —CH₂CH₂CH₃ | —CH₂CH₂CH₃ | 668 | 92.0 |
| Compound 139 | CH₃CH₂I/CH₃I | —CH₃ | —CH₂CH₃ | 626 | 92.4 |
| Compound 140 | Allyl bromide | H | 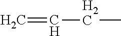 | 624 | 92.2 |
| Compound 141 | Propargyl bromide | H | 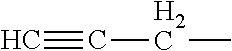 | 622 | 92.2 |
| Compound 142 | CH₃OBr | H | —OCH₃ | 614 | 92.1 |
| Compound 143 | CH₃OBr/CH₃I | —CH₃ | —OCH₃ | 628 | 92.3 |

Remark: Carbon atom with * is (R)-configuration

### Embodiment 22

### Preparation of (S)—N-(1-(3-fluorobenzyl)-1H-indazol-5-yl)-6-(5-(1-(amino)-2-(methylsulfonyl)ethyl)furan-2-yl)quinazolin-4-amine

Method A: The process is the same as the process in the method A in embodiment 18, except that the raw material, compound of embodiment 1 was replaced by compound of embodiment 5, and the product was numbered as Compound 144.

m/z (M+1)⁺: 557; e.e value: 93.0% [(S):96.5%, (R): 3.5%]

Method B: The process is the same as the process in the method B in embodiment 18, except that the raw material, compound of embodiment 15 was replaced by compound of embodiment 12.

According to the preparation methods in embodiment 10, the compound obtained in embodiment 22, as the starting material, was used to react with reagents to prepare the following compound:

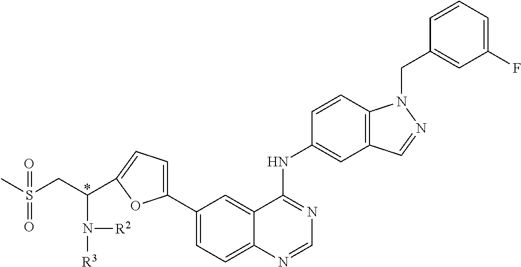

| | | | | m/z | e.e. |
|---|---|---|---|---|---|
| Number | Reagent | R² = | R³ = | (M + 1)⁺ | value % |
| Compound 145 | CH₃I | H | —CH₃ | 571 | 93.1 |
| Compound 146 | CH₃CH₂I | H | —CH₂CH₃ | 585 | 93.0 |
| Compound 147 | CH₃CH₂CH₂I | H | —CH₂CH₂CH₃ | 599 | 93.2 |
| Compound 148 | Cyclopropyl- methylbromide | H | 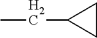 | 611 | 93.0 |
| Compound 149 | CH₃I | —CH₃ | —CH₃ | 585 | 93.1 |
| Compound 150 | CH₃CH₂I | —CH₂CH₃ | —CH₂CH₃ | 613 | 93.1 |
| Compound 151 | CH₃CH₂CH₂I | —CH₂CH₂CH₃ | —CH₂CH₂CH₃ | 641 | 93.2 |
| Compound 152 | CH₃CH₂I/CH₃I | —CH₃ | —CH₂CH₃ | 599 | 93.0 |
| Compound 153 | Allyl bromide | H | 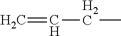 | 597 | 93.0 |
| Compound 154 | Propargyl bromide | H | 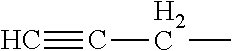 | 595 | 93.0 |
| Compound 155 | CH₃OBr | H | —OCH₃ | 587 | 93.1 |
| Compound 156 | CH₃OBr/CH₃I | —CH₃ | —OCH₃ | 601 | 93.0 |

Remark: Carbon atom with * is (S)-configuration

### Test 1

### Evaluation of Antitumor Activity In Vitro

### Experiment Method: SRB

### Cell Strains: A431; MCF-7

Experiment Design: Cells were incubated with different concentrations of compounds for 72 hours. The inhibition of cell proliferation by the compounds was evaluated by SRB, followed by calculation of the inhibition rate. Then, value of IC₅₀ was calculated by using-Logit method base on the inhibition rate and used to compare in vitro antitumor activity of the compounds.

Method for Calculating the Inhibition Rate: Inhibition rate (%)=(OD value of control group−OD value of drug group)/OD value of control group×100%

### Experiment Results:

| | IC₅₀ (nm) | |
|---|---|---|
| Number | A431 (72 h) | MCF-7 (72 h) |
| Lapatinib (positive compound) | 643.1 | 8468 |
| Compound 92 | 422 | 9336 |
| Compound 93 | 459.6 | 8861 |
| Compound 94 | 652.5 | 7853 |
| Compound 95 | 452.8 | 7900 |
| Compound 96 | 578.4 | 6700 |
| Compound 97 | 734.1 | 6666 |
| Compound 98 | 677.2 | 7658 |
| Compound 99 | 758.8 | 5922 |
| Compound 100 | 488.6 | 9896 |
| Compound 101 | 588.3 | 10426 |
| Compound 102 | 456.9 | 8900 |
| Compound 103 | 478.8 | 8824 |
| Compound 104 | 632.2 | 9004 |
| Compound 105 | 663.7 | 7865 |
| Compound 106 | 469.1 | 7800 |
| Compound 107 | 578.4 | 6766 |
| Compound 108 | 528.9 | 5900 |
| Compound 109 | 755.8 | 6211 |
| Compound 110 | 767.7 | 5669 |
| Compound 111 | 699.4 | 6823 |
| Compound 112 | 855.3 | 6218 |
| Compound 113 | 758.2 | 7066 |
| Compound 114 | 488.9 | 7900 |
| Compound 115 | 535.7 | 7580 |
| Compound 116 | 440.7 | 4800 |
| Compound 117 | 635.5 | 7626 |
| Compound 118 | 524.6 | 6687 |
| Compound 119 | 587.8 | 6121 |
| Compound 120 | 658.1 | 7562 |
| Compound 121 | 465.0 | 6387 |
| Compound 122 | 399.8 | 5968 |
| Compound 123 | 498.8 | 6328 |
| Compound 124 | 684.7 | 7980 |
| Compound 125 | 523.0 | 7764 |
| Compound 126 | 487.9 | 7764 |
| Compound 127 | 521.4 | 5889 |
| Compound 128 | 672.2 | 6336 |
| Compound 129 | 666.8 | 9711 |
| Compound 130 | 536.3 | 8588 |
| Compound 131 | 582.1 | 8525 |
| Compound 132 | 388.7 | 9907 |
| Compound 133 | 554.4 | 7887 |
| Compound 134 | 579.5 | 6510 |
| Compound 135 | 488.6 | 6190 |
| Compound 136 | 448.7 | 5430 |
| Compound 137 | 589.2 | 5470 |
| Compound 138 | 426.3 | 4599 |
| Compound 139 | 452.4 | 4130 |
| Compound 140 | 647.7 | 4020 |
| Compound 141 | 485.9 | 7624 |
| Compound 142 | 499.5 | 7865 |
| Compound 143 | 788.2 | 5090 |
| Compound 144 | 710.6 | 4970 |
| Compound 145 | 581.3 | 8240 |
| Compound 146 | 489.1 | 5460 |
| Compound 147 | 405.7 | 8010 |
| Compound 148 | 442.3 | 7001 |
| Compound 149 | 348.9 | 7230 |
| Compound 150 | 624.1 | 6674 |
| Compound 151 | 637.2 | 6080 |
| Compound 152 | 600.8 | 6540 |
| Compound 153 | 857.2 | 5870 |
| Compound 154 | 624.3 | 4897 |
| Compound 155 | 479.6 | 8588 |
| Compound 156 | 427.6 | 5520 |

### Test 2

Effect of compound 97(±), compound 97, compound 110 and Lapatinib (positive control) on xenografts in nude mice with lung cancer Calu-3 Remark: “±” means the compound is racemic.

### 1. Summary

Evaluation and comparison of effect of compound 97(±), compound 97, compound 110, and Lapatinib (positive control) on xenografts in nude mice with lung cancer Calu-3. Compound 97, compound 110 and Lapatinib all inhibited the growth of human lung cancer Calu-3 significantly. The efficiency in descending order is compound 110, compound 97(±), compound 97, Lapatinib. Compound 110 can cause most tumors in mice to shrink. Mice can well tolerate all the compounds mentioned above.

### 2. Purpose of the Experiment

The purpose of the experiment is to evaluate and compare the effect of compound 97(±), compound 97, compound 110, Lapatinib on xenografts in nude mice with lung cancer Calu-3.

### 3. Animals of the Experiment

BALB/cA-nude mice, 6-7 weeks, ♀, purchased from ShangHai Slac laboratory animal Co., Ltd.

Certificate NO.: SCXK (Shanghai) 2007-0005. Feeding condition: SPF grade.

### 4. Experimental Procedures

Calu-3 cells were injected subcutaneously into nude mice. Animals were divided into groups (d0) randomly after tumors have grown to 150-300 mm³. Dosage and dosage regimen design can be seen in table 1. Tumors volume was measured 2-3 times per week. Mice were weighed and data were recorded. Calculation formula for tumor volume (V) is as below:

V=½×a×b², wherein, a and b represent length and width respectively.

### 5. Experimental Results

Compound 97, compound 110 and Lapatinib all inhibited the growth of human lung cancer Calu-3 significantly. The efficiency in descending order is compound 110, compound 97(±), compound 97, Lapatinib. Compound 110 can cause ⅚ of the tumors in mice to shrink, with tumor in one mouse shrinking more than 50% in volume. Mice can well tolerate all the compounds mentioned above.

TABLE 1
Effect of Compound 97(±), Compound 97, Compound 110, Lapatinib on Xenografts in Nude Mice with Lung
Cancer Calu-3.

| | | Admin- | Initial Tumor | | Final Tumor | | Relative | | | Final Tumor | Relative | The | |
|---|---|---|---|---|---|---|---|---|---|---|---|---|---|
| | | istra- | Volume | | Volume | | Tumor | | % | Inhibition | Value (inal | Reces- | Mouse |
| | Drug | tion | (mm³) | | (mm³) | | Volume | | T/C | Rate (%) | vs. Control) | sion/ | Num- |
| Group | Cycle | Route | D0 | SEM | D16 | SEM | D16 | SEM | D16 | D16 | D16 | Group | ber |
| Vehicle | QD × 17 | PO | 268.0 | ±19.3 | 887.7 | ±109.3 | 3.4 | ±0.4 | 100 | 0 | — | 0 | 10 |
| Compound 97(±) | QD × 17 | PO | 255.2 | ±28.8 | 279.6 | ±30.9 | 1.1 | ±0.1 | 33 | 67 | 0.001 | 0 | 6 |
| 146.5 mg/kg | | | | | | | | | | | | | |
| Compound 97(+) | QD × 17 | PO | 291.2 | ±14.8 | 525.4 | ±104.1 | 1.9 | ±0.5 | 56 | 44 | 0.037 | 0 | 6 |
| 146.6 mg/kg | | | | | | | | | | | | | |
| Compound 110(−) | QD × 17 | PO | 249.1 | ±19.3 | 187.4 | ±24.4 | 0.8 | ±0.1 | 23 | 77 | 0.000 | 1 | 6 |
| 146.7 mg/kg | | | | | | | | | | | | | |
| Lapatinib | QD × 17 | PO | 262.3 | ±21.6 | 526.5 | ±98.1 | 2.0 | ±0.3 | 58 | 42 | 0.027 | 0 | 6 |
| 146.7 mg/kg | | | | | | | | | | | | | |

D0: Time of the first administration;
D16: The 17^{th} day after administration;
n = 10 for control group;
n = 6 for drug group;
T represents mean tumor volume of drug group;
C represents mean tumor volume of control group.

1. A compound of formula (I) or a pharmaceutically acceptable salt thereof, 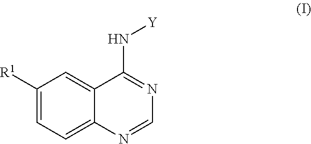
wherein R¹ represents 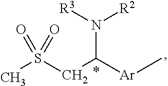
in which Ar is selected from the group consisting of unsubstituted or substituted furan and unsubstituted or substituted thiazole, the substituent is selected from the group consisting of halogen, C₁₋₄ alkyl, and C₁₋₄ alkoxy, and the number of substituents is 1 or 2; R² and R³ are independently selected from the group consisting of: hydrogen, alkyl, alkenyl, alkynyl, alkoxy, alkoxy alkyl, cycloalkyl, and cycloalkyl alkyl,
Y is selected from the group consisting of phenyl and 1H-indazol substituted by R⁴ and R⁵;
wherein R⁴ is selected from the group consisting of benzyl, halogenated-, dihalogenated- or trihalogenated-benzyl, benzyloxy, halogenated-, and dihalogenated- or trihalogenated-benzyloxy; and
R⁵ is selected from the group consisting of hydrogen, hydroxy, halogen atoms, C₁₋₄ alkyl, C₁₋₄ alkoxy, amino, cyano, and trifluoromethyl; and
the carbon atom with * is a chiral carbon atom; and wherein the compound is at least enantiomerically enriched with the (R) or (S) enantiomer.

2. The compound of claim 1 or a pharmaceutically acceptable salt thereof, wherein Ar is selected from the group consisting of unsubstituted furan and unsubstituted thiazole.

3. The compound of claim 1 or a pharmaceutically acceptable salt thereof, wherein R² and R³ are independently selected from the group consisting of: hydrogen, C₁₋₄ alkyl, C₂₋₅ alkenyl, C₂₋₅ alkynyl, C₁₋₄ alkoxy, C₁₋₄ alkoxy C₁₋₄ alkyl, C₃₋₈ cycloalkyl, and C₃₋₈ cycloalkyl-C₁₋₄ alkyl.

4. The compound of claim 1 or a pharmaceutically acceptable salt thereof, wherein R⁴ is selected from the group consisting of benzyl, halogenated-benzyl, and halogenated-benzyloxy.

5. The compound of claim 1 or a pharmaceutically acceptable salt thereof, wherein the compound exists in the form of a single enantiomer of (R).

6. The compound of claim 1 or a pharmaceutically acceptable salt thereof, wherein the compound is enriched in one enantiomer of (R), preferably and the percentage of enantiomer of (R) is ≧90%.

7. The compound of claim 1 or a pharmaceutically acceptable salt thereof, wherein the compound is selected from the group consisting of:
(R)—N-(4-(3-fluorobenzyl-oxy)-3-chlorophenyl)-6-(5-(1-(amino)-2-(methylsulfonyl)ethyl)furan-2-yl)quinazolin-4-amine;
(S)—N-(4-(3-fluorobenzyl-oxy)-3-chlorophenyl)-6-(5-(1-(amino)-2-(methylsulfonyl)ethyl)furan-2-yl)quinazolin-4-amine;
(R)—N-(4-(3-fluorobenzyl-oxy)-3-chlorophenyl)-6-(5-(1-(methylamino)-2-(methylsulfonyl)ethyl)furan-2-yl)quinazolin-4-amine;
(R)—N-(4-(3-fluorobenzyl-oxy)-3-chlorophenyl)-6-(5-(1-(ethylamino)-2-(methylsulfonyl)ethyl)furan-2-yl)quinazolin-4-amine;
(R)—N-(4-(3-fluorobenzyl-oxy)-3-chlorophenyl)-6-(5-(1-(propyl-amino)-2-(methyl-sulfonyl)ethyl)furan-2-yl)quinazolin-4-amine;
(R)—N-(4-(3-fluorobenzyl-oxy)-3-chlorophenyl)-6-(5-(1-(cyclopropyl-methyl-amino)-2-(methylsulfonyl)ethyl)furan-2-yl)quinazolin-4-amine;
(R)—N-(4-(3-fluorobenzyl-oxy)-3-chlorophenyl)-6-(5-(1-(N,N-dimethyl-amino)-2-(methylsulfonyl)ethyl)furan-2-yl)quinazolin-4-amine;
(R)—N-(4-(3-fluorobenzyl-oxy)-3-chlorophenyl)-6-(5-(1-(N,N-diethyl-amino)-2-(methylsulfonyl)ethyl)furan-2-yl)quinazolin-4-amine;
(R)—N-(4-(3-fluorobenzyl-oxy)-3-chlorophenyl)-6-(5-(1-(N,N-dipropyl-amino)-2-(methylsulfonyl)ethyl)furan-2-yl)quinazolin-4-amine;
(R)—N-(4-(3-fluorobenzyloxy)-3-chlorophenyl)-6-(5-(1-(N-methyl, N-ethyl-amino)-2-(methylsulfonyl)ethyl)furan-2-yl)quinazolin-4-amine;
(R)—N-(4-(3-fluorobenzyl-oxy)-3-chlorophenyl)-6-(5-(1-(allyl-amino)-2-(methylsulfonyl)ethyl)furan-2-yl)quinazolin-4-amine;
(R)—N-(4-(3-fluorobenzyl-oxy)-3-chlorophenyl)-6-(5-(1-(propargyl-amino)-2-(methyl-sulfonyl)ethyl)furan-2-yl)quinazolin-4-amine;
(S)—N-(4-(3-fluorobenzyl-oxy)-3-chlorphenyl)-6-(5-(1-(methyl-amino)-2-(methyl-sulfonyl)ethyl)furan-2-yl)quinazolin-4-amine;
(S)—N-(4-(3-fluorobenzyl-oxy)-3-chlorophenyl)-6-(5-(1-(ethyl-amino)-2-(methyl-sulfonyl)ethyl)furan-2-yl)quinazolin-4-amine;
(S)—N-(4-(3-fluorobenzyl-oxy)-3-chlorophenyl)-6-(5-(1-(propyl-amino)-2-(methyl-sulfonyl)ethyl)furan-2-yl)quinazolin-4-amine;
(S)—N-(4-(3-fluorobenzyl-oxy)-3-chlorophenyl)-6-(5-(1-(cyclopropyl-methyl-amino)-2-(methylsulfonyl)ethyl)furan-2-yl)quinazolin-4-amine;
(S)—N-(4-(3-fluorobenzyl-oxy)-3-chlorophenyl)-6-(5-(1-(N,N-dimethyl-amino)-2-(methylsulfonyl)ethyl)furan-2-yl)quinazolin-4-amine;
(S)—N-(4-(3-fluorobenzyl-oxy)-3-chlorophenyl)-6-(5-(1-(N,N-diethyl-amino)-2-(methylsulfonyl)ethyl)furan-2-yl)quinazolin-4-amine;
(S)—N-(4-(3-fluorobenzyl-oxy)-3-chlorophenyl)-6-(5-(1-(N,N-dipropyl-amino)-2-(methylsulfonyl)ethyl)furan-2-yl)quinazolin-4-amine;
(S)—N-(4-(3-fluorobenzyl-oxy)-3-chlorophenyl)-6-(5-(1-(N-methyl, N-ethyl-amino)-2-(methylsulfonyl)ethyl)furan-2-yl)quinazolin-4-amine;
(S)—N-(4-(3-fluorobenzyl-oxy)-3-chlorophenyl)-6-(5-(1-(allyl-amino)-2-(methylsulfonyl)ethyl)furan-2-yl)quinazolin-4-amine;
(S)—N-(4-(3-fluorobenzyl-oxy)-3-chlorophenyl)-6-(5-(1-(propargyl-amino)-2-(methyl-sulfonyl)ethyl)furan-2-yl)quinazolin-4-amine;
(S)—N-(1-(3-fluorobenzyl)-1H-indazol-5-yl)-6-(5-(1-(amino)-2-(methylsulfonyl)ethyl)furan-2-yl)quinazolin-4-amine;
(S)—N-(1-(3-fluorobenzyl)-1H-indazol-5-yl)-6-(5-(1-(methylamino)-2-(methylsulfonyl)ethyl)furan-2-yl)quinazolin-4-amine;
(S)—N-(1-(3-fluorobenzyl)-1H-indazol-5-yl)-6-(5-(1-(ethyl-amino)-2-(methylsulfonyl)ethyl)furan-2-yl)quinazolin-4-amine;
(S)—N-(1-(3-fluorobenzyl)-1H-indazol-5-yl)-6-(5-(1-(propyl-amino)-2-(methylsulfonyl)ethyl)furan-2-yl)quinazolin-4-amine;
(S)—N-(1-(3-fluorobenzyl)-1H-indazol-5-yl)-6-(5-(1-(N,N-dimethyl-amino)-2-(methyl-sulfonyl)ethyl)furan-2-yl)quinazolin-4-amine;
(S)—N-(1-(3-fluorobenzyl)-1H-indazol-5-yl)-6-(5-(1-(cyclopropyl-methyl-amino)-2-(methylsulfonyl)ethyl)furan-2-yl)quinazolin-4-amine;
(S)—N-(1-(3-fluorobenzyl)-1H-indazol-5-yl)-6-(5-(1-(allyl-amino)-2-(methylsulfonyl)ethyl)furan-2-yl)quinazolin-4-amine; and
(S)—N-(1-(3-fluorobenzyl)-1H-indazol-5-yl)-6-(5-(1-(propargyl-amino)-2-(methyl-sulfonyl)ethyl)furan-2-yl)quinazolin-4-amine.

8. A method for preparing the compound of formula (I) in claim 1, comprising:
(1) reacting the compound of formula (II) with tert-butylsulfinamide to prepare the compound of formula (III); 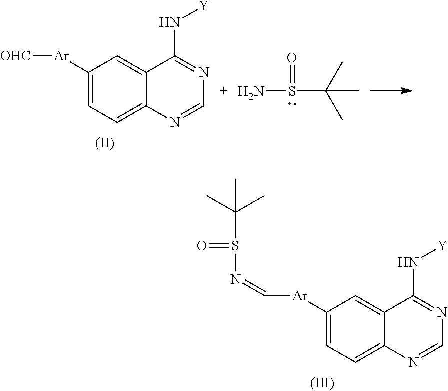
(2) reacting the compound of formula (III) with the compound of formula (IV) to prepare the compound of formula (V); 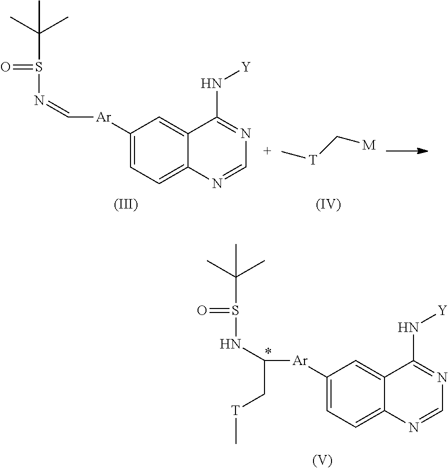
(3) generating the compound of formula (VI) from the compound of formula (V); 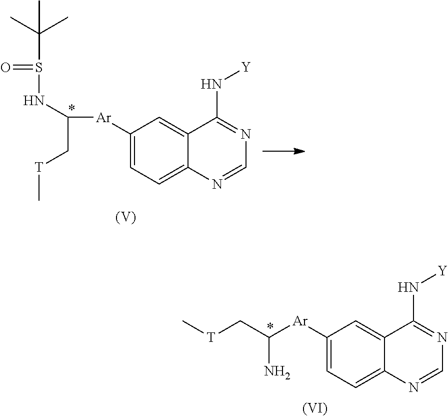
(4) reacting the compound of formula (VI) with reagent R²-L and R³-L to prepare the compound of formula (VII); and 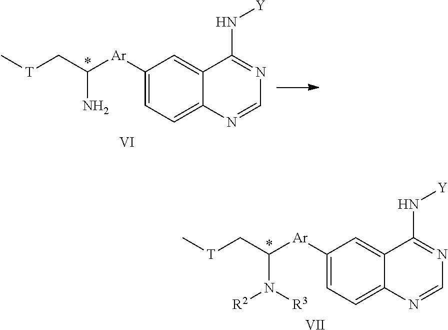
(5) converting the compound of formula (VII) to the compound of formula (I); 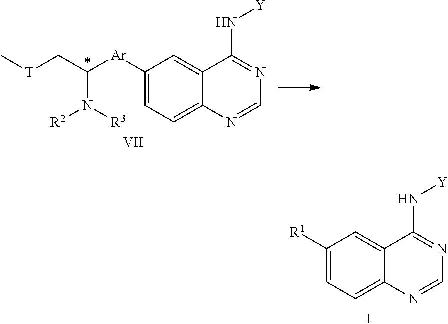
wherein R¹ represents 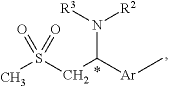
Ar is selected from the group consisting of unsubstituted or substituted furan and unsubstituted or substituted thiazole, the substituent is selected from the group consisting of halogen, C₁₋₄ alkyl, and C₁₋₄ alkoxy, and the number of substituents is 1 or 2; R² and R³ are independently selected from the group consisting of hydrogen, alkyl, alkenyl, alkenyl, alkoxy, alkoxy alkyl, cycloalkyl, and cycloalkyl alkyl,
Y is selected from the group consisting of phenyl and 1H-indazol substituted by R⁴ and R⁵;
wherein R⁴ is selected from the group consisting of benzyl, halogenated- dihalogenated- or trihalogenated-benzyl, benzyloxy, halogenated-, and dihalogenated- or trihalogenated-benzyloxy; and
R⁵ is selected from the group consisting of hydrogen, hydroxy, halogen atoms, C₁₋₄ alkyl, C₁₋₄ alkoxy, amino, cyano, and trifluoromethyl; and
carbon atom with * is a chiral carbon atom;
T is sulfur atom or sulfinyl;
tert-butylsulfinamide is optically pure;
M is alkali metal ion or halogenated-alkaline earth metal ions, selecting from the group consisting of Li⁺, Na⁺, K⁺, [MgCl]⁺ and [MgBr]⁺; and
L is a leaving group, selecting from the group consisting of halogen atoms and sulfonyloxyl group.

9. The method of claim 8, wherein the generating of compound of formula (VI) is carried out under acidic conditions, and wherein the acid is selected from the group consisting of hydrochloric acid, sulfuric acid, nitric acid, phosphoric acid, trifluoroacetic acid and the combinations thereof.

10. A method for preparing the compound of formula (I) in claim 1, comprising:
(1) reacting the compound of formula (II) with tert-butylsulfinamide to prepare the compound of formula (III); 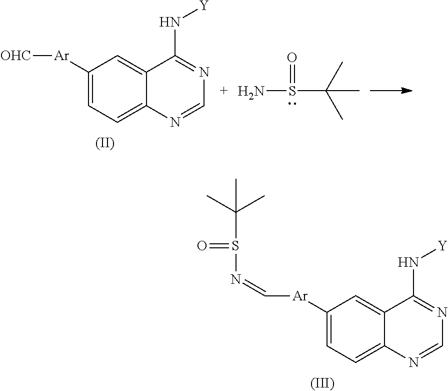
(2) reacting the compound of formula (III) with the compound of formula (A) to prepare the compound of formula (B); 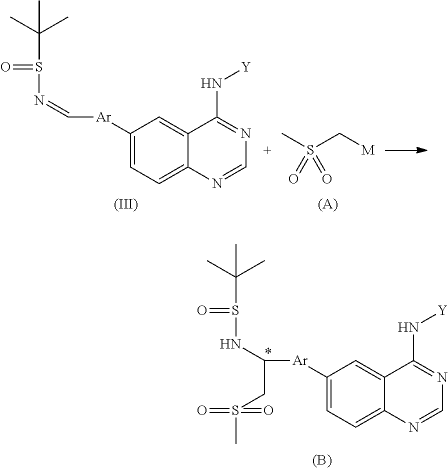
(3) generating the compound of formula (C) from the compound of formula (B); and 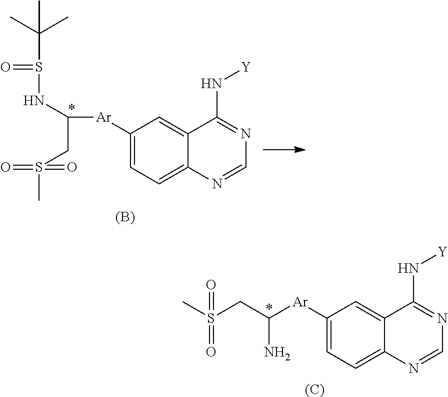
(4) reacting the compound of general formula (C) with reagent R²-L and R³-L to prepare the compound of formula (I); 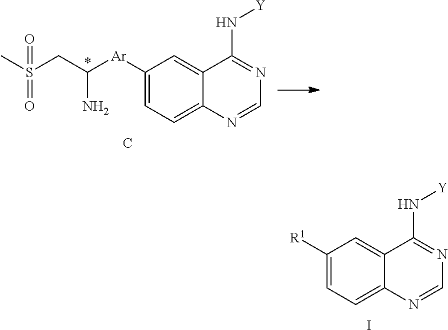
wherein tert-butylsulfinamide is optically pure;
R¹ represents 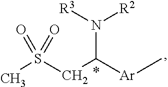
Ar is selected from the group consisting of unsubstituted or substituted furan and unsubstituted or substituted thiazole, the substituent is selected from the group consisting of halogen, C₁₋₄ alkyl, and C₁₋₄ alkoxy, and the number of substituents is 1 or 2; R² and R³ are independently selected from the group consisting of hydrogen, alkyl, alkenyl, alkynyl, alkoxy, alkoxy alkyl, cycloalkyl, and cycloalkyl alkyl,
Y is selected from the group consisting of phenyl and 1H-indazol substituted by R⁴ and R⁵;
wherein R⁴ is selected from the group consisting of benzyl, halogenated-, dihalogenated- or trihalogenated-benzyl, benzyloxy, halogenated-, and dihalogenated- or trihalogenated-benzyloxy; and
R⁵ is selected from the group consisting of hydrogen, hydroxy, halogen atoms, C₁₋₄ alkyl, C₁₋₄ alkoxy, amino, cyano, and trifluoromethyl;
the carbon atom with * is a chiral carbon atom;
M is alkali metal ion or halogenated-alkaline earth metal ions, selected from the group consisting of Li⁺, Na⁺, K⁺, [MgCl]⁺ and [MgBr]⁺; and
L is a leaving group, selecting from the group consisting of halogen atoms and sulfonyloxyl group.

11. A compound of formula (VII), 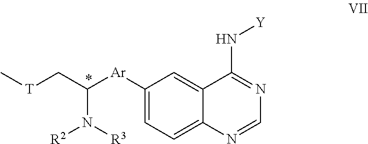
wherein Ar is selected from the group consisting of unsubstituted or substituted furan and unsubstituted or substituted thiazole, the substituent is selected from the group consisting of halogen, C₁₋₄ alkyl, and C₁₋₄ alkoxy, and the number of substituents is 1 or 2;
R² and R³ are independently selected from the group consisting of hydrogen, alkyl, alkenyl, alkynyl, alkoxy, alkoxy alkyl, cycloalkyl, and cycloalkyl alkyl;
Y is selected from the group consisting of phenyl and 1H-indazol substituted by R⁴ and R⁵;
wherein R⁴ is selected from the group consisting of benzyl, halogenated-, dihalogenated- or trihalogenated-benzyl, benzyloxy, halogenated-, and dihalogenated- or trihalogenated-benzyloxy; and
R⁵ is selected from the group consisting of hydrogen, hydroxy, halogen atoms, C₁₋₄ alkyl, C₁₋₄ alkoxy, amino, cyano, and trifluoromethyl;
the carbon atom with * is a chiral carbon atom, wherein the compound is at least enantiomerically enriched with the (R) or (S) enantiomer; and
T is sulfur atom or sulfinyl.

12. The compound of claim 11, wherein the compound is selected from the group consisting of:
(R)—N-(4-(3-fluorobenzy-oxy)-3-chlorophenyl)-6-(5-(1-(amino)-2-(methylthio)ethyl)furan-2-yl)quinazolin-4-amine;
(R)—N-(4-(3-fluorobenzyl-oxy)-3-chlorophenyl)-6-(5-(1-(methyl-amino)-2-(methyl-thio)ethyl)furan-2-yl)quinazolin-4-amine;
(R)—N-(4-(3-fluorobenzyl-oxy)-3-chlorophenyl)-6-(5-(1-(ethyl-amino)-2-(methyl-thio)ethyl)furan-2-yl)quinazolin-4-amine;
(R)—N-(4-(3-fluorobenzyl-oxy)-3-chlorophenyl)-6-(5-(1-(propyl-amino)-2-(methyl-thio)ethyl)furan-2-yl)quinazolin-4-amine;
(R)—N-(4-(3-fluorobenzyl-oxy)-3-chlorophenyl)-6-(5-(1-(cyclopropyl-methyl-amino)-2-(methylthio)ethyl)furan-2-yl)quinazolin-4-amine;
(R)—N-(4-(3-fluorobenzyl-oxy)-3-chlorophenyl)-6-(5-(1-(N,N-dimethyl-amino)-2-(methylthio)ethyl)furan-2-yl)quinazolin-4-amine;
(R)—N-(4-(3-fluorobenzyloxy)-3-chlorophenyl)-6-(5-(1-(N,N-diethyl-amino)-2-(methylthio)ethyl)furan-2-yl)quinazolin-4-amine;
(R)—N-(4-(3-fluorobenzyl-oxy)-3-chlorophenyl)-6-(5-(1-(N,N-dipropyl-amino)-2-(methylthio)ethyl)furan-2-yl)quinazolin-4-amine;
(R)—N-(4-(3-fluorobenzyl-oxy)-3-chlorophenyl)-6-(5-(1-(N-methyl, N-ethyl-amino)-2-(methylthio)ethyl)furan-2-yl)quinazolin-4-amine;
(R)—N-(4-(3-fluorobenzyl-oxy)-3-chlorophenyl)-6-(5-(1-(allyl-amino)-2-(methyl-thio)ethyl)furan-2-yl)quinazolin-4-amine;
(R)—N-(4-(3-fluorobenzyl-oxy)-3-chlorophenyl)-6-(5-(1-(propargyl-amino)-2-(methylthio)ethyl)furan-2-yl)quinazolin-4-amine;
(R)—N-(4-(3-fluorobenzyl-oxy)-3-chlorophenyl)-6-(5-(1-(methyl-amino)-2-(methyl-sulfinyl)ethyl)furan-2-yl)quinazolin-4-amine;
(R)—N-(4-(3-fluorobenzyl-oxy)-3-chlorophenyl)-6-(5-(1-(ethyl-amino)-2-(methyl-sulfinyl)ethyl)furan-2-yl)quinazolin-4-amine;
(R)—N-(4-(3-fluorobenzyl-oxy)-3-chlorophenyl)-6-(5-(1-(propyl-amino)-2-(methyl-sulfinyl)ethyl)furan-2-yl)quinazolin-4-amine;
(R)—N-(4-(3-fluorobenzyl-oxy)-3-chlorophenyl)-6-(5-(1-(cyclopropyl-methyl-amino)-2-(methylsulfinyl)ethyl)furan-2-yl)quinazolin-4-amine;
(R)—N-(4-(3-fluorobenzyl-oxy)-3-chlorophenyl)-6-(5-(1-(N,N-dimethyl-amino)-2-(methylsulfinyl)ethyl)furan-2-yl)quinazolin-4-amine;
(R)—N-(4-(3-fluorobenzyl-oxy)-3-chlorophenyl)-6-(5-(1-(N,N-diethyl-amino)-2-(methylsulfinyl)ethyl)furan-2-yl)quinazolin-4-amine;
(R)—N-(4-(3-fluorobenzyl-oxy)-3-chlorophenyl)-6-(5-(1-(N,N-dipropyl-amino)-2-(methylsulfinyl)ethyl)furan-2-yl)quinazolin-4-amine;
(R)—N-(4-(3-fluorobenzyloxy)-3-chlorophenyl)-6-(5-(1-(N-methyl, N-ethyl-amino)-2-(methylsulfinyl)ethyl)furan-2-yl)quinazolin-4-amine;
(R)—N-(4-(3-fluorobenzyl-oxy)-3-chlorophenyl)-6-(5-(1-(allyl-amino)-2-(methylsulfinyl)ethyl)furan-2-yl)quinazolin-4-amine;
(R)—N-(4-(3-fluorobenzyl-oxy)-3-chlorophenyl)-6-(5-(1-(propargyl-amino)-2-(methylsulfinyl)ethyl)furan-2-yl)quinazolin-4-amine;
(S)—N-(4-(3-fluorobenzyl-oxy)-3-chlorophenyl)-6-(5-(1-(amino)-2-(methylthio)ethyl)furan-2-yl)quinazolin-4-amine;
(S)—N-(4-(3-fluorobenzyl-oxy)-3-chlorophenyl)-6-(5-(1-(methyl-amino)-2-(methyl-thio)ethyl)furan-2-yl)quinazolin-4-amine;
(S)—N-(4-(3-fluorobenzyl-oxy)-3-chlorophenyl)-6-(5-(1-(ethyl-amino)-2-(methyl-thio)ethyl)furan-2-yl)quinazolin-4-amine;
(S)—N-(4-(3-fluorobenzyl-oxy)-3-chlorophenyl)-6-(5-(1-(propyl-amino)-2-(methylthio)ethyl)furan-2-yl)quinazolin-4-amine;
(S)—N-(4-(3-fluorobenzyl-oxy)-3-chlorophenyl)-6-(5-(1-(cyclopropyl-methyl-amino)-2-(methylthio)ethyl)furan-2-yl)quinazolin-4-amine;
(S)—N-(4-(3-fluorobenzyl-oxy)-3-chlorophenyl)-6-(5-(1-(N,N-dimethyl-amino)-2-(methylthio)ethyl)furan-2-yl)quinazolin-4-amine;
(S)—N-(4-(3-fluorobenzyl-oxy)-3-chlorophenyl)-6-(5-(1-(N,N-diethyl-amino)-2-(methylthio)ethyl)furan-2-yl)quinazolin-4-amine;
(S)—N-(4-(3-fluorobenzyloxy)-3-chlorophenyl)-6-(5-(1-(N,N-dipropyl-amino)-2-(methylthio)ethyl)furan-2-yl)quinazolin-4-amine;
(S)—N-(4-(3-fluorobenzyl-oxy)-3-chlorophenyl)-6-(5-(1-(N-methyl, N-ethyl-amino)-2-(methylthio)ethyl)furan-2-yl)quinazolin-4-amine;
(S)—N-(4-(3-fluorobenzyl-oxy)-3-chlorophenyl)-6-(5-(1-(allyl-amino)-2-(methylthio)ethyl)furan-2-yl)quinazolin-4-amine;
(S)—N-(4-(3-fluorobenzyl-oxy)-3-chlorophenyl)-6-(5-(1-(propargyl-amino)-2-(methylthio)ethyl)furan-2-yl)quinazolin-4-amine;
(S)—N-(1-(3-fluorobenzyl)-1H-indazol-5-yl)-6-(5-(1-(methyl-amino)-2-(methylthio)ethyl)furan-2-yl)quinazolin-4-amine;
(S)—N-(1-(3-fluorobenzyl)-1H-indazol-5-yl)-6-(5-(1-(ethyl-amino)-2-(methylthio)ethyl)furan-2-yl)quinazolin-4-amine;
(S)—N-(1-(3-fluorobenzyl)-1H-indazol-5-yl)-6-(5-(1-(propyl-amino)-2-(methylthio)ethyl)furan-2-yl)quinazolin-4-amine;
(S)—N-(1-(3-fluorobenzyl)-1H-indazol-5-yl)-6-(5-(1-(N,N-dimethyl-amino)-2-(methylthio)ethyl)furan-2-yl)quinazolin-4-amine;
(S)—N-(1-(3-fluorobenzyl)-1H-indazol-5-yl)-6-(5-(1-(cyclopropyl-methyl-amino)-2-(methylthio)ethyl)furan-2-yl)quinazolin-4-amine;
(S)—N-(1-(3-fluorobenzyl)-1H-indazol-5-yl)-6-(5-(1-(N-methyl, N-ethyl-amino)-2-(methylthio)ethyl)furan-2-yl)quinazolin-4-amine;
(S)—N-(1-(3-fluorobenzyl)-1H-indazol-5-yl)-6-(5-(1-(ally-amino)-2-(methylthio)ethyl)furan-2-yl)quinazolin-4-amine;
(S)—N-(1-(3-fluorobenzyl)-1H-indazol-5-yl)-6-(5-(1-(propargyl-amino)-2-(methyl-thio)ethyl)furan-2-yl)quinazolin-4-amine;
(S)—N-(4-(3-fluorobenzyl-oxy)-3-chlorophenyl)-6-(5-(1-(methyl-amino)-2-(methyl-sulfinyl)ethyl)furan-2-yl)quinazolin-4-amine;
(S)—N-(4-(3-fluorobenzyl-oxy)-3-chlorophenyl)-6-(5-(1-(ethyl-amino)-2-(methyl-sulfinyl)ethyl)furan-2-yl)quinazolin-4-amine;
(S)—N-(4-(3-fluorobenzyl-oxy)-3-chlorophenyl)-6-(5-(1-(propyl-amino)-2-(methyl-sulfinyl)ethyl)furan-2-yl)quinazolin-4-amine;
(S)—N-(4-(3-fluorobenzyl-oxy)-3-chlorophenyl)-6-(5-(1-(cyclopropyl-methyl-amino)-2-(methylsulfinyl)ethyl)furan-2-yl)quinazolin-4-amine;
(S)—N-(4-(3-fluorobenzyl-oxy)-3-chlorophenyl)-6-(5-(1-(N,N-dimethyl-amino)-2-(methylsulfinyl)ethyl)furan-2-yl)quinazolin-4-amine;
(S)—N-(4-(3-fluorobenzyl-oxy)-3-chlorophenyl)-6-(5-(1-(N,N-diethyl-amino)-2-(methylsulfinyl)ethyl)furan-2-yl)quinazolin-4-amine;
(S)—N-(4-(3-fluorobenzyl-oxy)-3-chlorophenyl)-6-(5-(1-(N,N-dipropyl-amino)-2-(methylsulfinyl)ethyl)furan-2-yl)quinazolin-4-amine;
(S)—N-(4-(3-fluorobenzyl-oxy)-3-chlorophenyl)-6-(5-(1-N-methyl, N-ethyl-amino)-2-(methylsulfinyl)ethyl)furan-2-yl)quinazolin-4-amine;
(S)—N-(4-(3-fluorobenzyl-oxy)-3-chlorophenyl)-6-(5-(1-(allyl-amino)-2-(methyl-sulfinyl)ethyl)furan-2-yl)quinazolin-4-amine; and
(S)—N-(4-(3-fluorobenzyl-oxy)-3-chlorophenyl)-6-(5-(1-(propargyl-amino)-2-(methylsulfinyl)ethyl)furan-2-yl)quinazolin-4-amine.

13. A pharmaceutical composition comprising a therapeutically effective amount of the compound of claim 1 or a pharmaceutically acceptable salt thereof and a pharmaceutically acceptable carrier.

14. A method of regulating c-erbB-2 or EGF-R protein tyrosine kinase comprising administering of the compound of formula (I) in claim 1 or a pharmaceutically acceptable salt thereof to a patient in need of the treatment.

15. A method of regulating c-erbB-2 or EGF-R protein tyrosine kinase comprising administering the pharmaceutical composition of claim 13 to a patient in need of the treatment.

16. A method for treating malignant tumor or psoriasis, comprising administering a therapeutically effective amount of the compound, or a pharmaceutically acceptable salt thereof, of claim 1 to a patient in need of the treatment.

17. The compound of claim 1 or a pharmaceutically acceptable salt thereof, wherein R² is selected from the group consisting of hydrogen, methyl, ethyl, and propyl.

18. The compound of claim 1 or a pharmaceutically acceptable salt thereof, wherein R³ is selected from the group consisting of hydrogen, methyl, ethyl, propyl, cyclopropylmethyl, allyl, propargyl, and methoxy.

19. The compound of claim 1 or a pharmaceutically acceptable salt thereof, wherein R² and R³ are methyl.

20. The compound of claim 1 or a pharmaceutically acceptable salt thereof, wherein Ar is unsubstituted furan.

21. The compound of claim 1 or a pharmaceutically acceptable salt thereof, wherein the compound exists in the form of a single enantiomer of (S).

22. The compound of claim 1 or a pharmaceutically acceptable salt thereof, wherein the percentage of (S) enantiomer is 90%.

23. The compound of claim 1 or a pharmaceutically acceptable salt thereof, wherein R⁴ is halogenated-benzyl or halogenated-benzyloxy.

24. The compound of claim 1 or a pharmaceutically acceptable salt thereof, wherein R⁵ is selected from the group consisting of hydrogen, halogen, C₁₋₄ alkyl and C₁₋₄ alkoxy.

25. The compound of claim 1 or a pharmaceutically acceptable salt thereof, wherein Y is phenyl substituted by chlorine and fluorobenzyloxy.

26. The compound of claim 26, or a pharmaceutically acceptable salt thereof, wherein Y is 3-chloro-4-((3-fluorobenzyl)oxy)phenyl.

27. The compound of claim 1, or a pharmaceutically acceptable salt thereof, wherein Y is indazol substituted by halogenated-benzyl.

28. The compound of claim 1, or a pharmaceutically acceptable salt thereof, wherein Y is N-(1-(3-fluorobenzyl)-1H-indazol.

29. The compound of claim 1, or a pharmaceutically acceptable salt thereof, wherein the compound is selected from the group consisting of:
(S)—N-(3-chloro-4-((3-fluorobenzyl)oxy)phenyl)-6-(5-(1-(methoxyamino)-2-(methylsulfonyl)ethyl)furan-2-yl)quinazolin-4-amine;
(S)—N-(3-chloro-4-((3-fluorobenzyl)oxy)phenyl)-6-(5-(1-(methoxy(methyl)amino)-2-(methylsulfonyl)ethyl)furan-2-yl)quinazolin-4-amine;
(R)—N-(3-chloro-4-((3-fluorobenzyl)oxy)phenyl)-6-(5-(1-(methoxyamino)-2-(methylsulfonyl)ethyl)furan-2-yl)quinazolin-4-amine;
(R)—N-(3-chloro-4-((3-fluorobenzyl)oxy)phenyl)-6-(5-(1-(methoxy(methyl)amino)-2-(methylsulfonyl)ethyl)furan-2-yl)quinazolin-4-amine;
(S)—N-(4-(3-fluorobenzyl-oxy)-3-chlorophenyl)-6-(5-(1-(amino)-2-(methylsulfonyl)ethyl)thiazole-2-yl)quinazolin-4-amine;
(S)—N-(3-chloro-4-((3-fluorobenzyl)oxy)phenyl)-6-(5-(1-(methylamino)-2-(methylsulfonyl)ethyl)thiazol-2-yl)quinazolin-4-amine;
(S)—N-(3-chloro-4-((3-fluorobenzyl)oxy)phenyl)-6-(5-(1-(ethylamino)-2-(methylsulfonyl)ethyl)thiazol-2-yl)quinazolin-4-amine;
(S)—N-(3-chloro-4-((3-fluorobenzyl)oxy)phenyl)-6-(5-(2-(methylsulfonyl)-1-(propylamino)ethyl)thiazol-2-yl)quinazolin-4-amine;
(S)—N-(3-chloro-4-((3-fluorobenzyl)oxy)phenyl)-6-(5-(1-((cyclopropylmethyl)amino)-2-(methylsulfonyl)ethyl)thiazol-2-yl)quinazolin-4-amine;
(S)—N-(3-chloro-4((3-fluorobenzyl)oxy)phenyl)-6-(5-(1-(dimethylamino)-2-(methylsulfonyl)ethyl)thiazol-2-yl)quinazolin-4-amine;
(S)—N-(3-chloro-4-((3-fluorobenzyl)oxy)phenyl)-6-(5-(1-(diethylamino)-2-(methylsulfonyl)ethyl)thiazol-2-yl)quinazolin-4-amine;
(S)—N-(3-chloro-4-((3-fluorobenzyl)oxy)phenyl)-6-(5-(1-(dipropylamino)-2-(methylsulfonyl)ethyl)thiazol-2-yl)quinazolin-4-amine;
(S)—N-(3-chloro-4-((3-fluorobenzyl)oxy)phenyl)-6-(5-(1-(ethyl(methyl)amino)-2-(methylsulfonyl)ethyl)thiazol-2-yl)quinazolin-4-amine;
(S)-6-(5-(1-(allylamino)-2-(methylsulfonyl)ethyl)thiazol-2-yl)-N-(3-chloro-4-((3-fluorobenzyl)oxy)phenyl)quinazolin-4-amine;
(S)—N-(3-chloro-4-((3-fluorobenzyl)oxy)phenyl)-6-(5-(2-(methylsulfonyl)-1-(prop-2-yn-1-ylamino)ethyl)thiazol-2-yl)quinazolin-4-amine;
(S)—N-(3-chloro-4-((3-fluorobenzyl)oxy)phenyl)-6-(5-(1-(methoxyamino)-2-(methylsulfonyl)ethyl)thiazol-2-yl)quinazolin-4-amine;
(S)—N-(3-chloro-4-((3-fluorobenzyl)oxy)phenyl)-6-(5-(1-(methoxy(methyl)amino)-2-(methylsulfonyl)ethyl)thiazol-2-yl)quinazolin-4-amine;
(R)—N-(4-(3-fluorobenzyl-oxy)-3-chlorophenyl)-6-(5-(1-(amino)-2-(methylsulfonyl)ethyl)thiazole-2-yl)quinazolin-4-amine;
(R)—N-(3-chloro-4-((3-fluorobenzyl)oxy)phenyl)-6-(5-(1-(methylamino)-2-(methylsulfonyl)ethyl)thiazol-2-yl)quinazolin-4-amine;
(R)—N-(3-chloro-4-((3-fluorobenzyl)oxy)phenyl)-6-(5-(1-(ethylamino)-2-(methylsulfonyl)ethyl)thiazol-2-yl)quinazolin-4-amine;
(R)—N-(3-chloro-4-((3-fluorobenzyl)oxy)phenyl)-6-(5-(2-(methylsulfonyl)-1-(propylamino)ethyl)thiazol-2-yl)quinazolin-4-amine;
(R)—N-(3-chloro-4-((3-fluorobenzyl)oxy)phenyl)-6-(5-(1-((cyclopropylmethyl)amino)-2-(methylsulfonyl)ethyl)thiazol-2-yl)quinazolin-4-amine;
(R)—N-(3-chloro-4-((3-fluorobenzyl)oxy)phenyl)-6-(5-(1-(dimethylamino)-2-(methylsulfonyl)ethyl)thiazol-2-yl)quinazolin-4-amine;
(R)—N-(3-chloro-4-((3-fluorobenzyl)oxy)phenyl)-6-(5-(1-(diethylamino)-2-(methylsulfonyl)ethyl)thiazol-2-yl)quinazolin-4-amine;
(R)—N-(3-chloro-4-((3-fluorobenzyl)oxy)phenyl)-6-(5-(1-(dipropylamino)-2-(methylsulfonyl)ethyl)thiazol-2-yl)quinazolin-4-amine;
(R)—N-(3-chloro-4-((3-fluorobenzyl)oxy)phenyl)-6-(5-(1-(ethyl(methyl)amino)-2-(methylsulfonyl)ethyl)thiazol-2-yl)quinazolin-4-amine;
(R)-6-(5-(1-(allylamino)-2-(methylsulfonyl)ethyl)thiazol-2-yl)-N-(3-chloro-4-((3-fluorobenzyl)oxy)phenyl)quinazolin-4-amine;
(R)—N-(3-chloro-4-((3-fluorobenzyl)oxy)phenyl)-6-(5-(2-(methylsulfonyl)-1-(prop-2-yn-1-ylamino)ethyl)thiazol-2-yl)quinazolin-4-amine;
(R)—N-(3-chloro-4-((3-fluorobenzyl)oxy)phenyl)-6-(5-(2-(methylsulfonyl)-1-(prop-2-yn-1-ylamino)ethyl)thiazol-2-yl)quinazolin-4-amine;
(R)—N-(3-chloro-4-((3-fluorobenzyl)oxy)phenyl)-6-(5-(1-(methoxyamino)-2-(methylsulfonyl)ethyl)thiazol-2-yl)quinazolin-4-amine;
(R)—N-(3-chloro-4-((3-fluorobenzyl)oxy)phenyl)-6-(5-(1-(methoxy(methyl)amino)-2-(methylsulfonyl)ethyl)thiazol-2-yl)quinazolin-4-amine;
(S)-6-(5-(1-(diethylamino)-2-(methylsulfonyl)ethyl)furan-2-yl)-N-(1-(3-fluorobenzyl)-1H-indazol-5-yl)quinazolin-4-amine;
(S)-6-(5-(1-(dipropylamino)-2-(methylsulfonyl)ethyl)furan-2-yl)-N-(1-(3-fluorobenzyl)-1H-indazol-5-yl)quinazolin-4-amine;
(S)-6-(5-(1-(ethyl(methyl)amino)-2-(methylsulfonyl)ethyl)furan-2-yl)-N-(1-(3-fluorobenzyl)-1H-indazol-5-yl)quinazolin-4-amine;
(S)—N-(1-(3-fluorobenzyl)-1H-indazol-5-yl)-6-(5-(1-(methoxyamino)-2-(methylsulfonyl)ethyl)furan-2-yl)quinazolin-4-amine; and
(S)—N-(1-(3-fluorobenzyl)-1H-indazol-5-yl)-6-(5-(1-(methoxy(methyl)amino)-2-(methylsulfonyl)ethyl)furan-2-yl)quinazolin-4-amine.

30. The compound of claim 1, or a pharmaceutically acceptable salt thereof, wherein the compound is selected from the group consisting of (S)—N-(4-(3-fluorobenzyloxy)-3-chlorophenyl)-6-(5-(1-(N,N-dimethyl-amino)-2-(methylsulfonyl)ethyl)furan-2-yl)quinazolin-4-amine, and (R)—N-(4-(3-fluorobenzyl-oxy)-3-chlorophenyl)-6-(5-(1-(N,N-dimethyl-amino)-2-(methylsulfonyl)ethyl)furan-2-yl)quinazolin-4-amine.

31. The method of claim 8, wherein tert-butylsulfinamide is enriched in R-enantiomer.

32. The method of claim 8, wherein tert-butylsulfinamide is enriched in S-enantiomer.

33. The method of claim 9, wherein the acid is hydrochloric acid.

34. The method of claim 8, wherein converting the compound of formula (VII) to the compound of formula (I) is by an oxidant, and wherein the oxidant is selected from the group consisting of chloroperoxybenzoic acid, peracetic acid, hydrogen peroxide and potassium monopersulfate.

35. The method of claim 34, wherein the oxidant is potassium monopersulfate.

36. The method of claim 10, wherein generating the compound of formula (C) from the compound of formula (B) is carried out under acidic conditions.

37. The method of claim 36, wherein the acid is selected from the group consisting of hydrochloric acid, sulfuric acid, nitric acid, phosphoric acid, trifluoroacetic acid and the combinations thereof.

38. The method of claim 37, wherein the acid is hydrochloric acid.

39. The compound of claim 11, wherein R² is selected from the group consisting of hydrogen, methyl, ethyl, and propyl.

40. The compound of claim 11, wherein R³ is selected from the group consisting of hydrogen, methyl, ethyl, propyl, cyclopropylmethyl, allyl, propargyl, and methoxy.

41. The compound of claim 11, wherein R⁴ is halogenated-benzyl or halogenated-benzyloxy.

42. The compound of claim 11, wherein R⁵ is selected from the group consisting of hydrogen, halogen, C₁₋₄ alkyl and C₁₋₄ alkoxy.

43. The compound of claim 11, wherein Y is 3-chloro-4-((3-fluorobenzyl)oxy)phenyl.

44. The compound of claim 11, wherein Y is N-(1-(3-fluorobenzyl)-1H-indazol.
